(19) United States
(12) Patent Application Publication
(10) Pub. No.: US 2023/0107049 A1
(43) Pub. Date: Apr. 6, 2023

(54) ANALYSIS OF MICROBIOME FOR DIAGNOSIS AND TREATING OF URINARY STONE DISEASE

(71) Applicants: THE CLEVELAND CLINIC FOUNDATION, Cleveland, OH (US); UNIVERSITY OF UTAH, CENTER FOR TECHNOLOGY & VENTURE COMMERCIALIZATION, Salt Lake City, UT (US)

(72) Inventors: Aaron Miller, Cleveland, OH (US); Denise Dearing, Salt Lake City, UT (US); Manoj Monga, Cleveland, OH (US)

(21) Appl. No.: 17/802,597

(22) PCT Filed: Feb. 26, 2021
(86) PCT No.: PCT/US2021/019759

§ 371 Date: Aug. 26, 2022

Related U.S. Application Data
(60) Provisional application No. 62/982,873, filed on Feb. 28, 2020.

(51) Int. Cl. C12Q 1/689; C12Q 1/06; A61K 35/74

(52) U.S. Cl. CPC C12Q 1/689; A61K 35/74; C12Q 1/06

(57) ABSTRACT

A method of determining the risk that a subject will develop urinary stone disease (USD) or hyperoxaluria is described. The method includes conducting a differential abundance analysis of the bacteria present in a stool and/or urine sample obtained from the subject, determining the ratio of bacteria associated with health to bacteria associated with USD or hyperoxaluria present in the subject’s stool and/or urine sample, and assigning a level of risk for developing USD or hyperoxaluria based on the ratio.

### CROSS REFERENCE TO RELATED APPLICATIONS

This application claims priority to U.S. Provisional Pat. Application No. 62/982,873, filed on Feb. 28, 2020, which is hereby incorporated by reference in its entirety.

### GOVERNMENT FUNDING

This invention was made with government support under DEB-1342615 awarded by the National Science Foundation and DK102277 awarded by National Institutes of Health. The government has certain rights in the invention.

### BACKGROUND

Oxalic acid is a dicarboxylic acid of the formula CO₂H—CO₂H. Oxalic acid exists primarily as oxalate in biological organisms, which is the salt form of oxalic acid. Oxalate is a compound endogenously produced in the liver as part of normal metabolism and is also absorbed in the intestine from oxalate-containing foods such as spinach, rhubarb, strawberries, cranberries, nuts, cocoa, chocolate, peanut and butter. Oxalate is a metabolic end product in humans and other mammals. It is excreted by the kidneys into the urine. When combined with calcium, oxalic acid produces an insoluble product, calcium oxalate, which is the most abundant compound found in kidney stones.

Because mammals do not synthesize enzymes that degrade oxalate, oxalate levels in an individual are normally held in check by excretion and low absorption of dietary oxalate. Elevated concentrations of oxalate are associated with a variety of pathologies, such as primary hyperoxaluria, enteric hyperoxaluria, and idiopathic hyperoxaluria. Increased oxalate can be caused by consuming too much oxalate from foods, by hyperabsorption of oxalate from the intestinal tract, and by abnormal oxalate production.

Hyperoxaluria, is defined as an excessive amount of oxalate in the urine, usually >45 mg/day, and is associated with a number of health problems related to the deposit of calcium oxalate in the kidney tissue (nephrocalcinosis) or urinary tract (e.g., kidney stones, urolithiasis, and nephrolithiasis). A direct link between intestinal bacteria and calcium oxalate kidney stone disease came following the discovery of Oxalobacter formigenes (O. formigenes). This commensal intestinal bacterial species utilizes oxalate as its primary nutrient source due to the expression of a specialized set of enzymes capable of rapidly degrading high concentrations of the compound, thus, preventing its absorption into circulation. However, numerous studies have investigated the colonization status of recurrent kidney stone formers and non-stone forming controls and shown that the absence of O. formigenes alone is not causative of stone disease as some recurrent stone formers are colonized while some non-stone formers are not. To date, the networks of bacteria that may be responsible for oxalate metabolism and/or inhibition of USD in humans have not been identified.

### SUMMARY

The present disclosure is based, at least in part, on a number of surprising findings. First, oxalate metabolism is associated with a diverse and consistent microbial network. Additionally, USD is more closely associated with the loss of bacteria that can protect against USD than with the acquisition of bacteria that can facilitate the onset of USD. Furthermore, there is considerable overlap between the bacteria associated with oxalate metabolism and the bacteria present in healthy individuals with no history of USD (FIG. 1). However, despite this overlap, it has been found that there are hundreds of bacteria species that are associated with either healthy subjects or subject’s having USD. By identifying and quantifying these specific bacteria present in a subject’s stool and/or urine, the subject’s risk for developing USD can be determined.

In one aspect, the present disclosure can include a method of determining the risk that a subject will develop urinary stone disease (USD) or hyperoxaluria, comprising conducting a differential abundance analysis of the bacteria present in a stool and/or urine sample obtained from the subject, determining a ratio of bacteria associated with health to bacteria associated with USD or hyperoxaluria present in the subject’s stool and/or urine sample, and assigning a level of risk for developing USD or hyperoxaluria based on the ratio.

In another aspect, the present disclosure can include a method of decreasing the risk that a subject will develop USD or hyperoxaluria, comprising conducting a differential abundance analysis of the bacteria present in a stool and/or urine sample obtained from the subject, determining the bacteria associated with health that are either missing or diminished in the subject’s stool and/or urine sample, and administering to the subject a composition comprising one or more of the missing or diminished bacteria.

In a further aspect, the present disclosure can include a method of guiding the treatment of USD or hyperoxaluria, comprising conducting a differential abundance analysis of the bacteria present in a stool and/or urine sample obtained from the subject, determining a ratio of bacteria associated with health to bacteria associated with USD or hyperoxaluria present in the subject’s stool and/or urine sample, assigning a level of severity of USD or hyperoxaluria based on the ratio, and providing treatment appropriate for the level of severity.

### BRIEF DESCRIPTION OF THE FIGURES

The foregoing and other features of the present disclosure will become apparent to those skilled in the art to which the present disclosure relates upon reading the following description with reference to the accompanying drawings, in which:

FIG. 1 shows a meta-analysis of all studies that have examined the whole gut microbiota in terms of the (operational taxonomic units) (OTUs) associated with oxalate metabolism and those that are enriched in either healthy or USD groups, summarized to genus-level taxonomy. Genera are ordered from those that are enriched the most often (top) to least often (bottom). Primary data sources were independently analyzed if available. An (*) indicates that primary data was not available.

FIG. 2 shows a chart that can be used to assess an individual’s USD risk based upon their microbial profile. Presented is the ratio of bacteria enriched in the healthy group (present at levels > 5 count) to bacteria enriched in the USD group (> 5 count). The further left an individual is, the lower their presumed stone risk. The further right, the higher their risk.

FIGS. 3(a)-(c) show the characterization of the whole gut microbiota between healthy and USD cohorts; (a) Phylum-level profile of the microbiota. Statistical analysis (t-test) reveals a significant reduction in the Tenericutes phylum in USD patients (p=0.012); (b) Beta diversity of the microbial community membership, based on an unweighted UniFrac analysis. Circles represent the multivariate homogeneity of dispersion around a centroid for each group comparison; (c) Beta diversity of microbial community structure, based on weighted UniFrac analysis. Circles represent the multivariate homogeneity of dispersion around a centroid for each group comparison. 2-way PERMANOVA analysis reveals a significant Gender*USD-status response for weighted (c) but not unweighted (b) UniFrac analysis (Weighted UniFrac — USD-status: p=0.071; Gender: p=0.221; Gender*USD-status: p=0.018; Unweighted UniFrac — USD-status: p=0.181; Gender: p=0.335; Gender*USD-status: p=0.589).

FIGS. 4(a)-(d) show the differential abundance analysis of functionally relevant bacteria between the USD and Healthy cohorts. (a) Total number of differentially abundant OTUs between groups (103 enriched in Healthy cohort; 62 enriched in USD cohort); (b) Colonization by Oxalobacter for each group (relative risk analysis p-value=0.06); (c) Relative abundance of Oxalobacter for each group (t-test p-value=0.06); (d) No. of co-occurrence interactions of bacteria that exhibit a significant positive correlation to Oxalobacter in each group.

FIG. 5 shows the quantification of the oxalate microbiome. Genera enriched in healthy individuals, or positively correlated to Oxalobacter sp. were compared to those genera stimulated by oxalate in Neotoma albigula. Gray indicates genera significantly enriched in the USD or Healthy groups, correlated to O. formigenes, or stimulated by oxalate, while black indicates non-significant associations.

FIGS. 6(a)-(b) show the phylum-level profile of the microbiome by USD-status; (A) Phylum profile by specimen-type of samples that only underwent molecular analysis; (B) Phylum profile comparing molecular only vs. samples that were cultured prior to molecular analysis.

FIGS. 7(a)-(d) show microbiome analysis by specimen type; (a) PCoA plots based on a weighted UniFrac analysis by specimen type, with each principal component listed with % of the dissimilarity explained by the coordinate. Statistical significance was determined by an Adonis with 999 permutations. Letters denote differences with p<0.05; (b)-(c) The differential abundance of OTUs by specimen type as assessed by a negative binomial Wald test. Listed are the total number of OTUs defined within the group, along with the number of OTUs enriched in each specimen type; (d) The average proportion of OTUs found in both stool and urine, or urine and stone by USD-status. There were no significant differences by group.

FIGS. 8(a)-(d) show microbiome analysis by technique; (a)-(b) PCoA plots based on a weighted UniFrac analysis by technique for bacterial analysis, with each principal component listed with % of the dissimilarity explained by the coordinate. Statistical significance was determined by an Adonis with 999 permutations. Letters denote differences with p<0.05. PS=“Paired Sample” and indicates the paired stone samples (molecular vs. culture); (c)-(d) The differential abundance of OTUs by technique as assessed by a negative binomial Wald test. Listed are the total number of OTUs defined within the group, along with the number of OTUs enriched in each specimen type by group.

FIGS. 9(a)-(d) show microbiome analysis by USD-status; (a)-(b) PCoA plots based on a weighted UniFrac analysis by USD-status, with each principal component listed with % of the dissimilarity explained by the coordinate. Statistical significance was determined by an ANOSIM with 999 permutations. Letters denote differences with p<0.05; (c)-(d) The differential abundance of OTUs by USD status as assessed by a negative binomial Wald test. Listed are the total number of OTUs defined within the group, along with the number of OTUs enriched in each specimen type by group.

FIGS. 10(a)-(b) show metrics associated with O. formigenes between healthy and USD groups; (a) Colonization rate of O. formigenes between groups. Significance was determined by a relative risk test, followed by a post-hoc Fisher’s exact test (p>0.05); (b) Relative abundance of O. formigenes. Significance was determined by a student’s t-test (p>0.05).

FIGS. 11(a)-(b) show urinary metabolomic data; (a) PCA plot of creatinine-normalized metabolite concentrations by group; (b) Metabolites significantly different between healthy and USD groups. The number of significantly different metabolites are indicated for each group

FIGS. 12(a)-(d) show microbe-metabolite interaction networks of microbes and metabolites significantly enriched in the healthy or USD groups, for the urine metabolome & both the urine and stool microbiome. Listed are the total number of interactions, number of metabolites involved, and number of bacteria involved; (a) Healthy, urine metabolome x urine microbiome; (b) Healthy, urine metabolome x stool microbiome; (c) USD, urine metabolome x urine microbiome; (d) USD, urine metabolome x stool microbiome.

FIG. 13 shows the meta-analysis of all studies that have examined the whole gut microbiota in terms of the OTUs associated with the oxalate-degrading microbial network (ODMN) or enriched/depleted in the USD groups, summarized to genus level taxonomy. Genera are ordered from those that are enriched the most often (top) to the least often (bottom). The box indicates the studies in humans. Primary data sources were independently analyzed if available.

FIGS. 14(a)-(d) show phylogenic diversity comparing techniques to examine the microbiota in urine and stone. Significant p-values are listed next to groups that exhibited a difference by technique. Significance was determined by a student’s t-test; (a) species richness; (b) evenness; (c) Shannon’s index; (d) phylogenetic diversity.

FIGS. 15(A)-(E) show microbial transplant plots. The plots are labeled with the number of days post-transplant: (A) 0 days; (B) 3 days; (C) 6 days; (D) 9 days; and (E) 12 days.

FIGS. 16(A)-(C) show urinary/oxalate metrics after antibiotic and/or diet treatment as indicated in FIG. 15. Each time point represents the average daily value for the 3-day interval; (A) shows urinary creatinine excretion; (B) shows total microbial oxalate metabolism (oxalate consumed minus oxalate excreted); and (C) shows urinary oxalate excretion (Urox). Letters indicate statistically significant differences either by Treatment group (in legend) or by time point (on x-axis) as determined by a repeated measures ANOVA and post-hoc Tukey’s honestly significant difference analysis.

FIG. 17 shows a graph with different combinations of bacteria that were grown in the presence of 50 mM oxalate in order to quantify the differences in oxalate metabolism between the groups. Groups were as follows (from left to right): oxalate degrading bacteria alone; oxalate- and formate- degrading bacteria; all functional groups listed in Table 2; all functional groups listed in Table 2 minus the oxalate-degrading bacteria; the whole N. albigula community. Letters (A, B, C) indicate statistical groups.

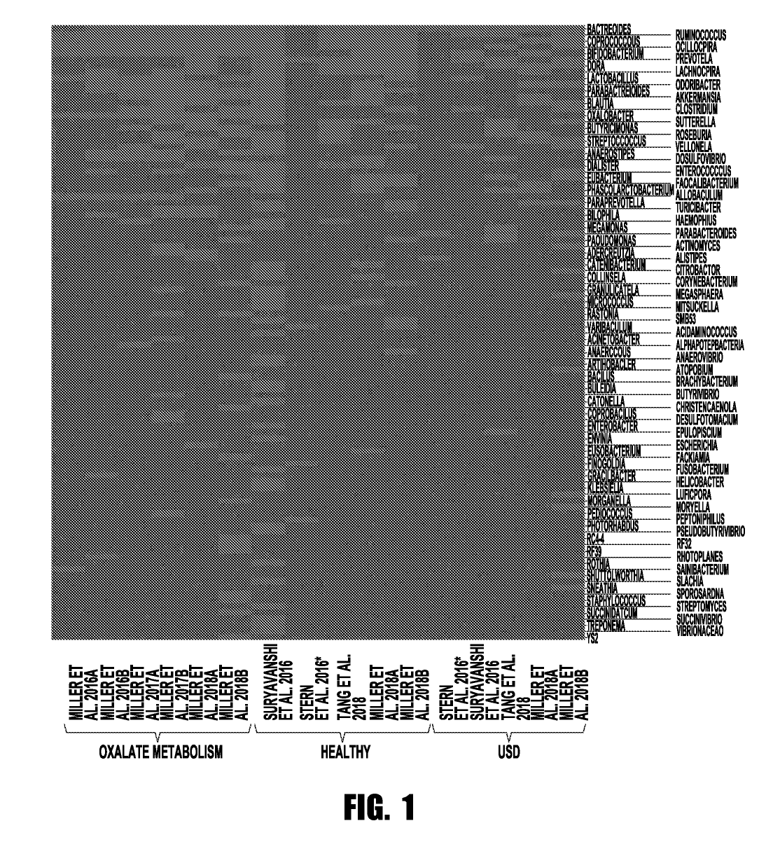

### DETAILED DESCRIPTION

### Definitions

Unless otherwise defined, all technical terms used herein have the same meaning as commonly understood by one of ordinary skill in the art to which the present disclosure pertains.

In the context of the present disclosure, the singular forms “a,” “an” and “the” can also include the plural forms, unless the context clearly indicates otherwise.

The terms “comprises” and/or “comprising,” as used herein, can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Also herein, where a range of numerical values is provided, it is understood that each intervening value is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

The terms “individual,” “subject,” and “patient” are used interchangeably herein irrespective of whether the subject has or is currently undergoing any form of treatment. As used herein, the term “subject” can refer to any vertebrate, including, but not limited to a mammal. In one instance the subject is a human.

As used herein, the term “diagnosis” can encompass determining the existence or nature of disease in a subject. As understood by those skilled in the art, a diagnosis does not indicate that it is certain that a subject has the disease, but rather that it is very likely that the subject has the disease. A diagnosis can be provided with varying levels of certainty, such as indicating that the presence of the disease is 70% likely, 85% likely, or 98% likely, for example. The term diagnosis, as used herein also encompasses determining the severity and probable outcome of disease or episode of disease or prospect of recovery, which is generally referred to as prognosis.

As used herein, the terms “treatment,” “treating,” and the like, can refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

As used herein, the terms “prevent” or “preventing” can refer to reducing the frequency or severity of a disease or disorder such as USD or hyperoxaluria. The term does not require an absolute preclusion of USD or hyperoxaluria. Rather, this term includes decreasing the chance that USD or hyperoxaluria will occur.

As used herein, the term “synbiotic” can refer to a combination of prebiotics and probiotics that synergistically promote gastrointestinal health.

As used herein, the term “taxonomic unit” can refer to a group of organisms that are considered similar enough to be treated as a separate unit. A taxonomic unit may comprise a family, genus, species or population within a species (e.g., strain), but is not limited as such.

As used herein the term “operational taxonomic unit” or “OTU” can refer to a group of microorganisms considered similar enough to be treated as a separate unit. An OTU may comprise a taxonomic family, genus or species but is not limited as such. In certain cases, the OTU may include a group of microorganisms treated as a unit based on e.g., a sequence identity of > 95%, > 90%, > 80%, or > 70% among at least a portion of a differentiating biomarker, such as the 16S rRNA gene.

### Methods of Risk Assessment

In one aspect, the present disclosure can provide a method of determining the risk that a subject will develop USD or hyperoxaluria. The method can include conducting a differential abundance analysis of the bacteria present in a stool and/or urine sample obtained from the subject, determining a ratio of bacteria associated with health to bacteria associated with USD or hyperoxaluria present in the subject’s stool and/or urine sample, and assigning a level of risk for developing USD or hyperoxaluria based on the ratio.

The term “urinary stone disease” or “USD” (also known as urolithiasis, nephrolithiasis, kidney stones, and nephrocalcinosis) can refer to the presence of stones and calcification within the urinary tract. Types of stones can include, for example, calcium oxalate (CaOx), calcium phosphate (CaP), uric acid, struvite (magnesium ammonium phosphate), cystine, and 2,8-dihydroxyadenine (2,8-DHA) stones.

The term “hyperoxaluria” can refer to secondary hyperoxaluria. Secondary hyperoxaluria is caused by increased dietary ingestion of oxalate, increased dietary ingestion of precursors of oxalate, or alteration in intestinal microflora. Secondary hyperoxaluira can be further classified as enteric hyperoxaluria, dietary hyperoxaluria, and idiopathic hyperoxaluria. Dietary hyperoxaluria refers to the increased consumption of high oxalate-content foods. Enteric hyperoxalruia refers to intestinal hyperabsorption of oxalate due to gastrointestinal disease. Idiopathic hyperoxaluria involves abnormal calcium handling by the gut, kidney, and bone.

In one instance, a stool sample can be collected from the subject and can be used to determine the risk that the subject will develop USD or hyperoxaluria. In another instance, a urine sample can be collected from the subject to determine the risk that the subject will develop USD or hyperoxaluria In a further instance, both a stool sample and a urine sample can be collected from the subject and can be used to determine the risk that a subject will develop USD or hyperoxaluria. Methods for collecting stool and urine samples are well known in the art. In some instances, the urine or stool sample can be collected using take home and mail-in kits. In other instances the urine or stool sample can be collected in a clinical setting. In certain instances, the stool and/or urine samples for analysis may be fresh or stored under suitable storage conditions. For instance, the stool and/or urine samples can be stored at low temperatures in order to prevent deterioration of the sample.

### Generating a Microbial Profile

As used herein, the term “microbial profile” can refer to the composition of the microbial community in a stool and/or urine sample, both qualitatively and quantitatively. The qualitative aspect can refer to the representative collection of species, genus groups, and/or other taxonomic groups present in a stool and/or urine sample. The qualitative aspect can refer to the relative abundance of each identified genus and species and/or other taxonomic groups present in a stool and/or urine sample.

Once a stool or and/or urine sample has been obtained, a microbial profile of the sample can be generated where the microbial profile can include both a qualitative and a quantitative component. The first step in generating a microbial profile can involve qualitatively identifying the representative collection of species, genus groups, and/or other taxonomic groups present in the sample. In some instances this can include carrying out DNA sequencing on the urine or stool sample. Methods for extracting and isolating DNA from urine and stool samples are known in the art and are routine. Any one of several commercially available DNA sequencing systems, such as the Illumina MiSeq or HiSeq platforms, the 454 pyrosequencing system, or the Ion Torrent system can be used to sequence the DNA extracted from the urine and/or stool samples.

In certain instances, DNA sequencing can include sequencing the bacterial DNA encoding for one or more rRNA gene sequences (e.g., 16 S, 23 S, 5 S rRNAs). For example, DNA sequencing can include sequencing the bacterial DNA encoding for one or more of the 16 S RNA hypervariable regions, V1 to V9, contained in a sample. In one aspect, the sequencing can include sequencing the bacterial DNA encoding for the V4 region of the 16 S rRNA gene.

The 16 S rRNA gene is particularly suitable as a biomarker for the identification and phylogenetic analysis of microorganisms. The 16 S rRNA gene offers several significant advantages as a biomarker. For example, some regions of the 16 S rRNA gene are highly conserved and universal PCR primer sets exist that can amplify the 16 S rRNA gene from the overwhelming majority of bacteria and Archea, respectively. The 16 S rRNA gene also includes regions that are less well conserved making it possible to identify taxons. Additionally, the 16 S rRNA gene is believed to have changed at a fairly constant rate during evolution, making it, in effect, an evolutionary clock with each nucleotide difference translating to an evolutionary time unit. The approximately 1500 bp sequence of the 16 S rRNA gene contains enough information to predict the identity and phylogeny of an organism with high precision. Furthermore, an extensive, rapidly growing database exists for this gene. For example, the ARB database (available on the world wide web at arb-home.de) contains over 25,000 aligned 16 S rRNA gene sequences.

When sequencing one or more hypervariable regions of 16 S DNA, the qualitative taxonomic profile can be determined using the following steps: (i) reads quality filtering and demultiplexing, (ii) paired reads merging, (iii) OTU (Operational taxonomic unit) clustering, e.g., at a 97% identity threshold, (iv) chimera filtering, and (v) taxonomy assignment.

The taxonomy assignment can be determined by comparing OTU representative sequences to databases such as SILVA database or Greengenes or to a customized reference table. In one instance the customized reference table can include the 16 S rRNA sequences of known bacterial species along with other identified bacterial sequences. The taxonomic identification steps can be performed using software such as QIIME or UPARSE, or the mothur taxonomy file database.

Shotgun metagenomic sequencing is an alternative approach to 16S sequencing, where all of the DNA present in the sample is fragmented and independently sequenced. The analysis steps performed to assess microbiome profiles from such data include three different methods used after read detection quality control procedures: (i) marker gene analyses (involving comparing each read to a reference database of taxonomically or phylogenetically informative sequences); (ii) a binning metagenomes method (including compositional binning, similarity binning, and fragment recruitment), and (iii) de novo assembly (reads are merged into contigs and blasted against reference databases to identify species).

### Differential Abundance Analysis

The term “relative abundance” can refer to the abundance of microorganisms of a particular OTU in a test sample compared to the abundance of microorganisms of the corresponding OTU in one or more non-diseased control samples. The “relative abundance” may be reflected in e.g., the number of isolated species corresponding to an OTU or the degree to which a biomarker specific for the OTU is present or expressed in a given sample. The relative abundance of a particular OTU in a sample can be determined using non-culture-based methods well known in the art. Non-culture based methods include sequence analysis of amplified polynucleotides specific for an OTU or a comparison of proteomics-based profiles in a sample reflecting the number and degree of polypeptide-based, lipid- based, polyssacharide-based or carbohydrate-based biomarkers characteristic of one or more OTUs present in the samples. Relative abundance or abundance of a taxa or OTU can be calculated with reference to all taxa/OTU detected, or with reference to some set of invariant taxa/OTUs.

The quantitative aspect of the microbial profile can be determined by differential abundance analysis. Differential abundance analysis can involve measuring the relative abundance of each OTU identified. An OTU table that gives the number of reads from each sample that is assigned to each OTU can be created by mapping the reads to OTUs. The relative abundance for a specific OTU in the sample can be defined as the ratio of number of reads mapping to the OTU to the total number of reads from the sample. The differential abundance analysis can provide the specific set of bacteria associated with healthy subjects that are enriched in the sample along with the specific set of bacteria associated with USD or hyperoxaluria that are enriched in the sample. In certain instances, a certain number of sequence read counts can indicate the presence of an OTU. For example, the presence of an OTU can be defined as having greater than five sequence read counts.

The data from the differential abundance analysis can be used to calculate a ratio of bacteria associated with healthy subjects (herein referred to as bacteria associated with health) that is present in the sample to bacteria associated with USD or hyperoxaluria that is present in the sample. In cases where the number of bacteria associated with health is greater than the number of bacteria associated with USD or hyperoxaluria, the ratio can be calculated as follows:

$$\text{ratio} \text{=} \frac{\# \text{of bacteria associated with health}}{\# \text{of bacteria associated with USD or hyperoxaluria} \text{.}}$$

In instances where the number of bacteria associated with USD or hyperoxaluria is greater than the number of bacteria associated with health, the ratio can be determined using the following calculation:

$$\text{Ratio=} \text{-} 1 \left[\frac{1}{\left(\frac{\# \text{of bacteria associated with health}}{\begin{matrix}\# \text{of bacteria associated with USD} \\ \text{or hyperoxaluria}\end{matrix}}\right)}\right]$$

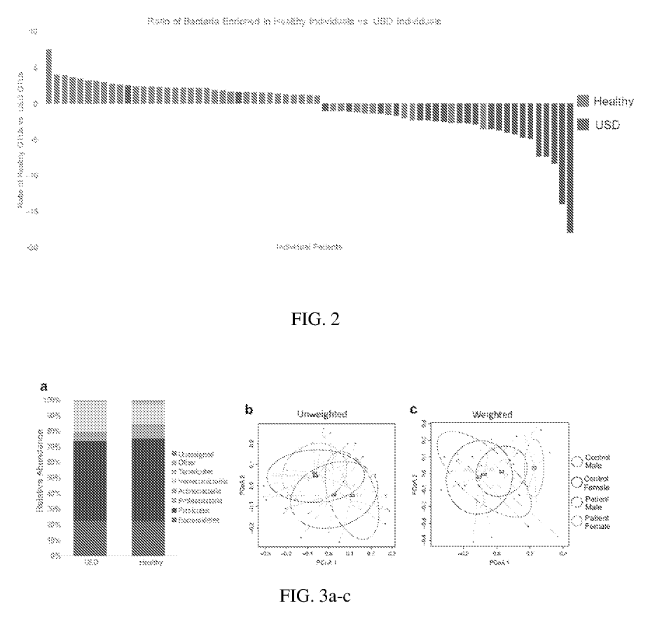

The ratio can then be compared to, e.g., a column chart as show in FIG. 2. As seen in FIG. 2, the column chart provides a range of ratios of healthy bacteria to USD bacteria. The further left a subject is on the chart (i.e., the higher the positive number), the lower the subject’s risk for urinary stone disease or hyperoxaluria is. The further right a subject is on the chart (i.e., the lower the negative number), the higher the subject’s risk for urinary stone disease or hyperoxaluria is. In one instance, a subject can be assigned a risk score based on their risk of developing USD or hyperoxaluria.

In certain instances, additional variables such as age, sex, diet, and previous stone types can be factored into a subject’s risk score for developing USD or hyperoxaluria.

### Bacterial Species

The methods described herein include the step of determining a ratio of bacteria associated with health to bacteria associated with USD or hyperoxaluria present in the subject’s stool and/or urine sample. The phrase “bacteria associated with health” can refer to those types of bacteria that exhibit significantly higher relative abundances in patients with no history of hyperoxaluria or USD compared to patients with hyperoxaluria or USD, respectively. Likewise, the phrase “bacteria associated with USD or hyperoxaluria” can refer to those types of bacteria that are present in a higher relative abundances in patients with USD or hyperoxaluria compared to patients with no history of USD.

Types of bacteria that are commonly associated with health include, but are not limited to bacteria in the genus Bacteroides (e.g., Bacteroides acidofaciens; Bacteroides vulgatus; Bacteroides dorei, Bacteroides betaiotamicron), bacteria in the genus Methanobrevibacter (e.g., Methanobrevibacter smithii), bacteria in the genus Coprococcus (e.g., Coprococcus comes), Lactobacillus helveticus, Lactobacillus plantarum, and Oxalobacter formigenes.

Bacteria species that are commonly associated with USD or hyperoxaluria include, but are not limited to bacteria from various genera of the Enterobacteriaceae family.

### Methods of Treating or Preventing USD or Hyperoxaluria

Another aspect of the disclosure includes a method of guiding the treatment of urinary stone disease or hyperoxaluria. The method includes conducting a differential abundance analysis of the bacteria present in a stool and/or urine sample obtained from the subject, determining a ratio of bacteria associated with health to bacteria associated with USD or hyperoxaluria present in the subject’s stool and/or urine sample, assigning a level of severity of USD or hyperoxaluria based on the ratio, and providing treatment appropriate for the level of severity.

One skilled in the art would understand that a medical professional can make treatment decisions based on an assessment of the severity of USD or hyperoxaluria. In certain instances, the treatment may include increased fluid intake or a change of diet. The change of diet may include limiting salt intake, decreasing sugar intake, eating less animal proteins (milk, egg, and fish), and avoiding foods high in oxalate levels such as spinach, bran flakes, rhubarb, beets, plums, chocolate, strawberries, tofu, almonds, potato chips, french fries, nuts and nut butters. In certain instances, medications can be provided including vitamin B-6 (pyridoxine) and thiazide diuretics. In certain instances, if a kidney stone is present and does not pass on its own, a procedure may be performed to remove the stone including shock wave lithotripsy, ureteroscopy, or percutaneous nephrolithotomy. Additionally, if hyperoxaluria is severe, kidney dialysis or organ transplantation (kidney or liver-kidney combination transplant) may be needed.

In another aspect, a method of decreasing the risk that a subject will develop USD or hyperoxaluria is provided. The method includes conducting a differential abundance analysis of the bacteria present in a stool and/or urine sample obtained from the subject, determining the bacteria associated with heath that are either missing or diminished in the subject’s stool and/or urine sample, and administering to the subject a composition comprising one or more of the missing or diminished bacteria.

The phrases “bacteria that are diminished” or “diminished bacteria” can refer to types of bacteria present in a lower amount such as at least 90%, 80%, 75%, or 70% of the amount present in a typical healthy individual.

In some embodiments, the bacterial compositions are administered to specific categories of subjects. In some embodiments, the pharmaceutical composition is administered to a subject who is on a low fat and/or sugar diet, while in other embodiments the pharmaceutical composition can be administered to a subject who is not taking an antibiotic compound.

### Bacterial Compositions

In some embodiments, the methods described herein can include administering one or more types of bacteria associated with health to the subject. These bacterial are administered as a composition, such as a pharmaceutically acceptable composition. Accordingly, another aspect of the present disclosure can include compositions for treating USD or hyperoxaluria as well as compositions for reducing a subject’s risk for USD or hyperoxaluria.

In one aspect, the composition can be formulated for a specific individual based on the subject’s microbial profile. For instance, the subject’s microbial profile can be analyzed to identify the types of bacterial (e.g., bacterial species) commonly associated with healthy individuals that are either missing or diminished in the subject’s microbial profile (hereinafter referred to as missing bacterial species). A composition can then be formulated to include, for example, one or more of the missing bacteria species.

In another aspect, the composition can be formulated to include one or more bacteria species that is associated with health but is not associated with subjects having USD or hyperoxaluria. For example, the composition can include one or more of the following: one or more species of bacteria falling under the genus Ruminococcus; one or more species of bacteria falling under the genus Enterobacter; Bacteroides acidofaciens; Bacteroides vulgatus; Bacteroides dorei; O. formigenes; Methanobrevibacter smithii; one or more species of bacteria falling under the genus Desulfovibrio; Bacteroides betaiotamicron; Coprococcus comes; Lactobacillus helveticus; and Lactobacillus plantarum.

In some instances, the composition can include one or more of the following: one or more species of bacteria falling under the genus Ruminococcus; one or more species of bacteria falling under the genus Enterobacter; Bacteroides acidofaciens; Bacteroides vulgatus; and Bacteroides dorei. In one specific instance, the composition can include one or more species of bacteria falling under the genus Ruminococcus; one or more species of bacteria falling under the genus Enterobacter; Bacteroides acidofaciens; Bacteroides vulgatus; and Bacteroides dorei.

In other instances, the composition can include one or more of the following: O. formigenes; Methanobrevibacter smithii; one or more species of bacteria falling under the genus Desulfovibrio; Bacteroides betaiotamicron; Coprococcus comes; Lactobacillus helveticus; and Lactobacillus plantarum. In one specific instance, the composition can include O. formigenes; Methanobrevibacter smithii; one or more species of bacteria falling under the genus Desulfovibrio; Bacteroides betaiotamicron; Coprococcus comes; Lactobacillus helveticus; and Lactobacillus plantarum.

In further instances, the composition can include any components necessary to support the bacteria species to be included in the composition. For example, the composition can include prebiotics. In one instance, the composition can include a prebiotics such as oxalate, formate, glucose, sucrose, galactose, aspartic acid, sodium acetate, mannose, fructose, or methyl-butyrate.

In other aspects, the composition can include additional compounds including those that e.g., stimulate oxalate metabolism and/or stimulate formate metabolism.

In some instances, the composition is a symbiotic composition that includes both probiotic bacteria and associated prebiotics.

The compositions described herein can be prepared from previously isolated bacterial strains. In some instances, the bacterial strains are held in public culture collections such as the American Type Culture Collection (ATCC). In other instances, the bacterial strains can be isolated from Neotoma albigula, including stool from Neotoma albigula. In further instances, the bacterial strains can be isolated from stools collected from humans without a history of USD.

The compositions can be formulated for delivery to the subject. In one example, the compositions can be administered as a pharmaceutical composition. In some instances a pharmaceutically acceptable excipient or carrier can be included in the pharmaceutical composition.

The term “excipient” can refer to any substance that enhances the absorption of any component of the preparation, i.e., bacterial strains, or that stabilizes said components and/or assists in the preparation of the pharmaceutical composition in that it provides consistency or a flavor that make it more palatable. Thus, the excipients can act to bind the components (for example, starches, sugars or celluloses), to sweeten, to provide a dye, to protect the active ingredient (for example, to insulate from air and/or humidity), to act as a filler in a pill, capsule or any form of presentation, to aid disintegration so as to facilitate dissolution of the components, etc., without excluding other excipients not listed in this paragraph. A “pharmacologically acceptable” excipient must not inhibit the activity of the compounds of the pharmaceutical formulation, that is, it must be compatible with the bacteria strains of the invention.

The “carrier” or “drug delivery vehicle” can be an inert substance. The function of the carrier/vehicle is to facilitate the incorporation of other compounds, and improve dosage and administration and/or confer consistency and form to the pharmaceutical composition. Therefore, the carrier/vehicle can be a substance used in the drug to dilute any component of the pharmaceutical composition of the present disclosure to a given volume or weight; or that allows for better dosage and administration and/or confers consistency and form to the drug.

The excipient and the carrier/vehicle can be pharmacologically acceptable, i.e., the excipient and carrier are permitted and have been demonstrated to be harmless to the subject to whom they are administered.

In one aspect, the format of the pharmaceutical composition can be adapted to the form of administration. The pharmaceutical composition can be formulated as a solid, semisolid or liquid preparation, such as a tablet, capsule, powder, granule, solution, suppository, gel, or microsphere. In certain instances, the pharmaceutical composition is in a form suitable for oral administration.

The dosages and dosage regimen in which the pharmaceutical compositions disclosed herein are administered can vary according to the dosage form, mode of administration, the condition being treated, and the particulars of the patient being treated.

### EXAMPLES

The following examples are for the purpose of illustration only and are not intended to limit the scope of the appended claims.

### Example 1: Isolation of Bacterial Strains

Isolations are done in strict anaerobic conditions using high throughput techniques. Source material is diluted to 10⁻⁷ in sterile serial dilutions, which approximates 1-5 cells per 100 ul. Aliquots of the dilution can be inoculated into e.g., five 96-well plates containing media designed to target the bacteria of interest (base media provided in Table 1; Table 2 includes additional carbon and energy substrates that can be added to the base media depending on the bacteria being isolated). The bacteria can then be incubated for up to one week. Purity and taxonomy of the isolate can be determined through 16S rRNA sequencing.

Table 1

| Component | Amount/L | Concentration |
|---|---|---|
| KH₂PO₄ | 100 mL | 100 mM |
| NaHCO₃ | 0.4 g | 4.8 mM |
| NH₄Cl | 1 g | |
| NaCl2 | 0.08 g | 1.37 mM |
| CaCl2 | 1 mL | 0.80% |
| Vitamin K (menadione) | 1 mL | 5.8 mM |
| FeSO₄ | 1 mL | 1.44 mM |
| Histidine Hematin Solution | 1 mL | 0.1% |
| Tween 80 | 2 mL | 0.05% |
| pH to 7* | | |
| DI H₂0 to 980 ml | | |
| Autoclave then move to anaerobic chamber | | |
| MgSO₄-7H₂0** | 1 mL | 0.008 mM |
| ATCC Vitamin Mix*** | 10 mL | 1% |
| ATCC Trace Mineral Mix*** | 10 mL | 1% |

*Bring pH up to 7 before autoclaving
**Filter sterilize
***Add to media in anaerobic media after autoclave

Table 2

Oxalate
Cellulose
Sucrose
D-Glucose
Aspartic Acid
Galactose
Formate (AMS bacteria)
Mannose
Fructose
Sodium Sulfate
Sodium Acetate
Methyl valeric/Butyric Acid
Glutamic Acid
Glyoxylic Acid

All pure cultures can be maintained in two ways. Backup cultures can be maintained in 15% glycerol at 80° C., under anaerobic conditions. The working culture can be maintained in mini-chemostats under active and stable growth.

### Example 2: Inhibition of USD

Given the prevalence of urinary stone disease and associated cost to healthcare systems, there exists a significant need for the development of novel treatment options for USD, as current options have not reduced the burden of this debilitating disease. A role for the urinary microbiome in recurrent USD has been established in the identification of differences in colonization status of patients and non-stone formers by the oxalotroph Oxalobacter formigenes, however recolonization attempts using this species have not resulted in overall decreased intestinal oxalate absorption and are not a viable treatment option. Given the symbiotic nature of the intestinal microbiome, it is highly likely that O. formigenes-induced oxalate homeostasis requires the collaboration of multiple bacterial species. The present work identifies a potential network of bacteria that together with O. formigenes act to maintain a healthy oxalate homeostasis. The identification of such a network provides a possible explanation for why recolonization attempts with O. formigenes alone have failed to result in non-stone forming environments in patients, and set the stage for recolonization studies using this multi-species network to develop more efficacious bacterial-based therapies to treat and prevent recurrent kidney stone disease.

The gut microbiota differences of patients with USD and co-habitating healthy controls were compared with emphasis on the microbial network associated with Oxalobacter to determine whether an oxalate-degrading microbiome plays a role in maintaining overall oxalate homeostasis in non-stone forming humans and whether an oxalate-degrading microbiome plays a role in stone disease in general.

### Results

In this study, 17 USD patients along with 17 live-in controls without USD patients were recruited. No participant had antibiotic exposure within at least one month prior to sample collection and none reported having undergone medical procedures and/or supplementation resulting in a significantly altered intestinal microbiome composition. Of all patients, 10 were confirmed CaOₓ stone formers either by stone analysis or via CT scan (Hounsfield unit measurement), two patients had uric acid stones, two patients had cystine stones, one patient had a struvite stone, while the stone type was unknown for two patients. The patient group consisted of 12 males and 5 females, had an average age of 58.0 +/- 12.1, average BMI of 30.52 +/- 5.42, and all but one had a significant history of stone recurrence. The control group consisted of five males and 12 females, had an average age of 59.18 +/-10.73 and average BMI of 25.85 +/- 5.87 (Table 3). Habitual dietary intake was collected from all patients and controls to determine a potential effect of varying diets on microbiome composition. Stool samples were collected from all participants for high throughput sequencing of the 16 S rRNA gene.

Table 3

| | | |
|---|---|---|
| Metric | Controls | Stone Patients |
| % Male | 29% | 71% |
| % Female | 71% | 29% |
| Age | 59.19 +/- 10.73 | 58.0 +/- 12.16 |
| BMI | 25.85 +/- 5.87 | 30.52 +/- 5.42 |
| % Obese | 18% | 42% |
| % Non-obese | 71% | 47% |
| % CaOx | NA | 53% |
| % Cystine | NA | 12% |
| % Uric Acid | NA | 12% |
| % Struvite | NA | 6% |
| USD history | NA | 2-30 yrs |

There were no differences for intake of nutrients and other dietary parameters between USD patients and controls (Table 4), including for macronutrient composition, estimations of potential renal acid load of diet, PRAL, net endogenous acid production, and NEAP. When stratifying by gender, there were no differences between male patients and controls nor between women patients and controls for any of the dietary parameters assessed. However, there were differences between men and women within the patient group, specifically: men had higher PRAL and NEAP than women (-0.67 vs. -12.68, P=0.043 from 2-tailed t-test; and 42.5 vs. 32.5 mEq/d, P=0.013 from 2-tailed t-test, respectively).

Table 4

| | Cases | Controls | P-value |
|---|---|---|---|
| Energy, kcals/d | 1,922 (1,437) | 2,085 (1,859) | 0.65 |
| PRAL¹ | -4.0 (0.0) | -5.2 (-6.0) | 0.82 |
| NEAP,² mEq/d | 40 (40) | 39 (39) | 0.79 |
| Protein, g/d (% of total kcals) | 79 (16%) | 87 (16%) | 0.60 |
| Fat, g/d (% of total kcals)³ | 75 (35%) | 84 (36%) | 0.52 |
| Carbohydrates, g/d (% of total kcals)³ | 238 (50% | 249 (48%) | 0.81 |
| Saturated fat, g/d (% of total kcals)³ | 23 (11%) | 26 (11%) | 0.58 |
| MUFA,⁴ g/d (% of total kcals) | 29 (14%) | 33 (14%)_ | 0.47 |
| PUFA,⁵ g/d (% of total kcals) | 16 (7.5%) | 18 (7.8%) | 0.50 |
| Added sugar, teaspoons/d | 13 (6.0) | 13 (10) | 0.82 |
| Fiber, g/d | 20 (20) | 22 (21) | 0.66 |
| Insoluble fiber, g/d | 14 (13) | 15 (14) | 0.46 |
| Soluble fiber, g/d | 7.3 (7.2) | 7.7 (7.3) | 0.75 |
| Fructose, g/d | 22 (18) | 23 (18) | 0.85 |
| Whole grains, servings/d | 0.92 (0.65) | 1.4 (1.2) | 0.17 |
| Refined grains, servings/d | 4.6 (2.8) | 4.5 (4.1) | 0.94 |
| Vegetables, servings/d | 5.3 (4.9) | 4.7 (4.5) | 0.57 |
| Dark green leafy vegetables, servings/d | 0.98 (0.50) | 0.80 (0.56) | 0.59 |
| Deep yellow vegetables, servings/d | 0.51 (0.47) | 0.43 (0.31) | 0.56 |
| Dry beans/peas, servings/d | 0.12 (0.09) | 0.12 (0.09) | 0.93 |
| White potatoes, servings/d | 0.48 (0.30) | 0.65 (0.44) | 0.44 |
| Other starchy vegetables, servings/d | 0.29 (0.22) | 0.26 (0.23) | 0.63 |
| Tomatoes, servings/d | 0.59 (0.43) | 0.59 (0.42) | 0.99 |
| Other vegetables, servings/d | 2.3 (1.9) | 1.8 (1.2) | 0.45 |
| Fruits, servings/d | 2.8 (2.3) | 3.1 (2.1) | 0.71 |
| Citrus, melon, berries, servings/d | 0.97 (0.80) | 1.4 (0.77) | 0.31 |
| Other fruits, servings/d | 1.8 (1.5) | 1.8 (1.4) | 0.87 |
| Dairy, servings/d | 1.5 (1.2) | 1.5 (1.3) | 0.86 |
| Dairy milk, servings/d | 0.56 (0.44) | 0.80 (0.45) | 0.35 |
| Yogurt, servings/d | 0.38 (0.26) | 0.29 (0.31) | 0.54 |
| Cheese, servings/d | 0.52 (0.27) | 0.44 (0.34) | 0.65 |
| Meats,⁶ounces/d | 4.0 (3.2) | 4.5 (4.0) | 0.58 |
| Fish, ounces/d | 0.63 (0.50) | 0.48 (0.46) | 0.34 |
| Nuts and seeds, ounces/d | 0.31 (0.30) | 0.56 (0.41) | 0.11 |
| Alcohol, drinks/d | 0.26 (0.15) | 0.37 (0.11) | 0.50 |
| Vitamin C, mg/d | 119 (125) | 135 (92) | 0.56 |
| Vitamin B6, mg/d | 1.9 (1.7) | 1.9 (1.8) | 0.81 |
| Calcium, mg/d | 852 (846) | 921 (769) | 0.66 |
| Magnesium, mg/d | 343 (323) | 395 (415) | 0.30 |
| Sodium, mg/d | 3,090 (2,567) | 3,298 (3,309) | 0.73 |
| Potassium, mg/d | 3,358 (3,200) | 3,691 (3,607) | 0.45 |

¹ PRAL, potential renal acid load of foods (validated calculation involving intake of protein, phosphorus, potassium, magnesium, and calcium)
² NEAP, net endogenous acid production (validated calculation involving intake of protein and potassium)
³ Percentages may not add up to 100% due to rounding
⁴ MUFA, monounsaturated fatty acids
⁵ PUFA, polyunsaturated fatty acids
⁶ Includes all flesh from all mammals, fowl, fish, seafood

The gut microbiota of patients with USD and healthy controls were characterized through sequencing. Sequencing of the V4 region of the 16S rRNA gene yielded a total of 2,207 ,898 high quality sequences from all 34 individuals with a total of 7,964 unique OTUs defined at the 97% similarity level. More than 99% of the OTUs were classified to the level of phylum, with 61% classified to the genus level. Both patients and controls were dominated by the Firmicutes phylum (∼52% of total for both), followed by the Bacteroidetes (22% for both). Taxonomic analysis revealed a significant reduction in the Tenericutes phylum present within the gut microbiota of patients vs. controls (FIG. 3a). Additionally, while there were no significant differences between groups in regards to alpha diversity (Table 5), there was a gender-specific difference in gut microbiota composition (beta-diversity) between patients and controls when the relative abundance of OTUs was taken into consideration (weighted UniFrac analysis) (FIGS. 3b,c).

Table 5

| | Margalef | Evenness | Shannon | Phylogenetic Diversity |
|---|---|---|---|---|
| Mean (USD) | 32.41 | 0.67 | 5.35 | 55.50 |
| Standard error | 5.61 | 0.03 | 0.26 | 8.77 |
| Mean (Healthy) | 37.95 | 0.72 | 5.94 | 64.53 |
| Standard error | 5.73 | 0.02 | 0.17 | 8.11 |
| T-test p-value | 0.49 | 0.15 | 0.07 | 0.46 |

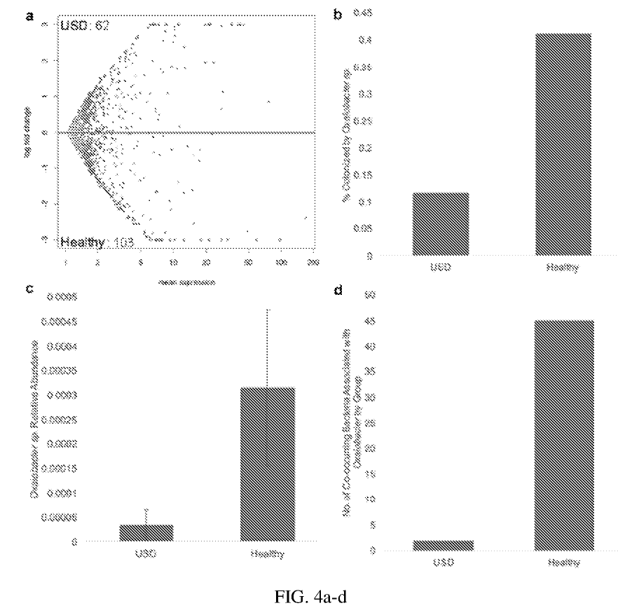

Differential abundance analysis revealed that 103 OTUs were significantly enriched in healthy individuals with only 62 enriched in patients (FIG. 4a, Table 6). While there was a trend towards higher relative abundance and colonization of O. formigenes in healthy individuals, the difference was not significant between groups (FIGS. 4b,c). A total of 149 OTUs exhibited a significant positive correlation with Oxalobacter sp. across the entire dataset (Table 7). In terms of the bacteria that correlated with Oxalobacter, there was an order of magnitude greater number of co-occurrence interactions in the healthy group compared to the USD group (FIG. 4d).

Table 6

OTUs differentially abundant between healthy and USD groups

| Lowest Assigned Taxonomy | Taxonomic Level | Group | No. of OTUs | FDR (range) |
|---|---|---|---|---|
| Akkermansia | genus | Healthy | 5 | <0.001 |
| Bacteroides | genus | Healthy | 14 | <0.001-0.027 |
| Bifidobacterium | genus | Healthy | 1 | <0.001 |
| Blautia | genus | Healthy | 3 | <0.001-0.047 |
| Catenibacterium | genus | Healthy | 1 | 0.041 |
| Christensenellaceae | family | Healthy | 1 | 0.006 |
| Clostridiaceae | family | Healthy | 1 | 0.023 |
| Clostridiales | order | Healthy | 10 | <0.001-0.003 |
| Collinsella | genus | Healthy | 3 | <0.001-0.028 |
| Coprococcus | genus | Healthy | 2 | 0.007-0.031 |
| Coriobacteriaceae | family | Healthy | 2 | 0.004-0.028 |
| Desulfovibrio | genus | Healthy | 1 | 0.002 |
| Dialister | genus | Healthy | 3 | <0.001-0.002 |
| Dorea | genus | Healthy | 1 | 0.005 |
| Enterobacteraceae | family | Healthy | 3 | <0.001-0.015 |
| Erysipelotrichaceae | family | Healthy | 2 | 0.002-0.029 |
| Eubacterium | genus | Healthy | 1 | <0.001 |
| Faecalibacterium | genus | Healthy | 2 | 0.003-0.032 |
| Lachnospira | genus | Healthy | 2 | 0.013-0.023 |
| Lachnospiraceae | family | Healthy | 8 | 0.005-0.041 |
| Methanobrevibacter | genus | Healthy | 1 | 0.008 |
| Odoribacter | genus | Healthy | 1 | 0.003 |
| Oscillospira | genus | Healthy | 2 | 0.006-0.046 |
| Parabacteroides | genus | Healthy | 4 | 0.002-0.028 |
| Phascolarctobacterium | genus | Healthy | 3 | <0.001-0.016 |
| Prevotella | genus | Healthy | 2 | <0.001-0.014 |
| Ruminococcaceae | family | Healthy | 21 | <0.001-0.04 |
| Ruminococcus (Lachnospiraceae) | genus | Healthy | 1 | 0.007 |
| Ruminococcus (Ruminococcaceae) | genus | Healthy | 1 | 0.033 |
| Sutterella | genus | Healthy | 1 | 0.033 |
| Clostridiaceae | family | USD | 2 | 0.027-0.043 |
| Enterobacteraceae | family | USD | 3 | 0.001-0.004 |
| Lachnospiraceae | family | USD | 13 | <0.001-0.048 |
| Mycoplasmataceae | family | USD | 2 | 0.006-0.02 |

Table 7

OTUs exhibiting a significantly positive correlation with Oxalobacter sp

| Lowest Assigned Taxonomy | Taxonomic Level | No. of OTUs | FDR (range) |
|---|---|---|---|
| Akkermansia | genus | 5 | 0.004-0.008 |
| Bacteroides | genus | 16 | 0.002-0.021 |
| Bifidobacterium | genus | 1 | <0.001 |
| Blautia | genus | 2 | <0.001-0.004 |
| Butyricimonas | genus | 1 | 0.004 |
| Christensellaceae | family | 4 | <0.001-0.029 |
| Clostridiales | order | 25 | <0.001-0.045 |
| Coprococcus | genus | 7 | <0.001-0.045 |
| Dorea | genus | 1 | <0.001 |
| Enterobacteraceae | family | 3 | 0.004-0.008 |
| Erysipelotrichaceae | family | 3 | <0.001-0.024 |
| Faecalibacterium | genus | 2 | 0.004-0.006 |
| Gemellaceae | family | 1 | 0.022 |
| Lachnospira | genus | 2 | 0.004-0.009 |
| Lachnospiraceae | family | 11 | 0.004-0.049 |
| Methanobrevibacter | genus | 1 | <0.001 |
| Mogibacteriaceae | family | 1 | 0.006 |
| Odoribacter | genus | 1 | 0.013 |
| Oscillospira | genus | 6 | <0.001-0.022 |
| Oxalobacter | genus | 3 | <0.001-0.005 |
| Phascolarctobacterium | genus | 4 | 0.004-0.008 |
| Prevotella | genus | 1 | 0.004 |
| Rikenellaceae | family | 2 | 0.004-0.012 |
| Roseburia | genus | 1 | <0.001 |
| Ruminococcaceae | family | 32 | <0.001-0.042 |
| Ruminococcus (Lachnospiraceae) | genus | 1 | 0.016 |
| Ruminococcus (Ruminococcaceae) | genus | 3 | 0.004-0.01 |
| Sutterella | genus | 2 | 0.004-0.028 |
| Turicibacter | genus | 1 | 0.004 |
| Unassigned | NA | 4 | 0.004 |
| YS2 | order | 1 | 0.004 |

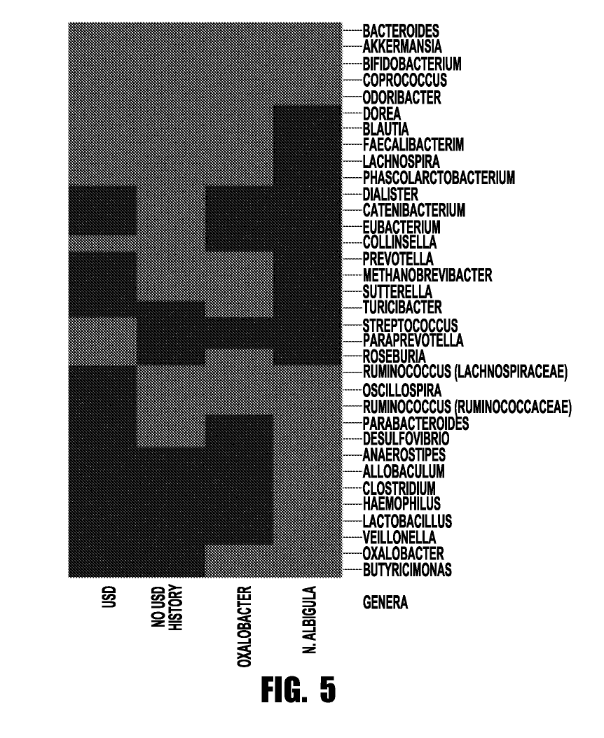

To further determine if healthy individuals harbored a more robust microbial network associated with oxalate metabolism than USD patients, we compared the list of bacteria enriched in the healthy and USD groups from this study to the list of bacteria exhibiting a significant positive correlation with Oxalobacter from N. albigula that were stimulated by oxalate (FIG. 5). Some taxa, such as Ruminococcus (from both the Ruminococcaceae and Lachnospiraceae families) and Oscillospira, had OTUs that were consistently present in healthy individuals and not in patients, were associated with the presence of Oxalobacter, and were stimulated by oxalate in the rodent model. However, other taxa, such as Bacteroides, Akkermansia, Bifidobacterium, Coprococcus, and Odoribacter, had OTUs in all four groups. Discussion

This study sought to address important gaps in knowledge regarding the role of the intestinal microbiome beyond O. formigenes and other oxalate-degrading bacteria in recurrent USD. A global approach to the analysis was taken rather than focusing on specific subsets of patients such as those with hyperoxaluria, in order to identify potential differences that exist across individuals suffering from USD. In the study the analysis was focused around microbial networks associated with oxalate metabolism.

Overall, a significant reduction in the Tenericutes phylum was found in all USD patients compared to the healthy population (FIG. 3a). Additionally, there was a gender-specific significant difference in community structure, but not in membership between the microbiota of patients and controls (FIGS. 3b,c), which may reflect the gender-based differences in stone risk. When looking at the differential abundance of specific OTUs within each of the groups, 62 OTUs were found to be enriched in patients vs. 103 OTUs enriched in non-stone forming controls. Interestingly, O. formigenes was conspicuously absent from the list of OTUs enriched in the controls, suggesting that other bacterial species are more important for the prevention of USD and/or oxalate homeostasis. Additionally, it was found that the number of co-occurrence interactions with bacteria associated with Oxalobacter discriminated patients from healthy controls more effectively than looking at the presence or absence of O. formigenes alone.

To identify bacterial taxa with importance in oxalate homeostasis, the list of bacteria enriched in healthy individuals and associated with Oxalobacter species (from this study) was compared to that of bacterial species stimulated by dietary oxalate in N. albigula. This herbivorous rodent is ideal to study microbial oxalate metabolism as the species has consumed a high oxalate diet in the wild for thousands of years and thus has not lost any bacteria due to laboratory rearing, drastic dietary changes, or antibiotic exposure, as is the case for laboratory rodents and humans. The comparison revealed two important points. First, some taxa, such as Ruminococcus (from both the Ruminococcaceae and Lachnospiraceae families) and Oscillospira, had OTUs that 1) were consistently present in healthy individuals and not in patients, 2) were associated with the presence of Oxalobacter, and 3) were stimulated by oxalate in rodents.

The second point revealed by our comparison is that some taxa, such as Bacteroides, Akkermansia, Bifidobacterium, Coprococcus, and Odoribacter, had OTUs stimulated in all compared groups. Thus, the data provides preliminary evidence that taxa previously associated with oxalate metabolism in rodents also convey a protective role against USD in humans. Adding weight to this is the finding that the OTUs that were different between the patient and control intestinal microbiota included taxa, such as Desulvovibrio and Methanobrevibacter, which engage in sulfate-reduction, methanogenesis, and acetogenesis. These bacterial species likely associate closely with O. formigenes as methanogens, acetogens, and sulfate-reducing bacteria utilize formate, the major by-product of oxalate metabolism by O. formigenes, as a source of carbon and energy. Similarly acetogens utilize CO₂, another major by-product from oxalate breakdown, to produce acetate a beneficial nutrient for the host and other microbes. The fact that O. formigenes does not express enzymes necessary for the assimilate formate or CO₂ increases the likelihood that it has to rely on other bacterial species for these functions. This underscores the likelihood that bacteria other than those directly involved in oxalate breakdown play a significant role in oxalate metabolism in vivo. This observation may further explain why recolonization with O. formigenes alone has only transient results.

It was also found that disturbances in the oxalate-degrading microbial network were also present in patients with non-oxalate stones. In total, at least five of the patients included in this study suffered from stones that were not calcium oxalate based, yet their microbiome showed dysbiosis that included members of the oxalate-degrading microbial network. While the sample number may be low, the data do suggest that disturbances in the intestinal microbiome including members associated with the oxalate-degrading microbial network identified here, may increase the risk for the development of other stone types.

The absence of dietary differences between cases and controls suggests that the observed differences in gut microbiota were related to the pathophysiology of stones and/or other factors and not to diet. While nutrient analyses of results from the C-DHQ-I did not include oxalate (due to limitations of the nutrient analysis software), surrogate measures of oxalate intake, including whole grains, vegetables (including dark green leafy vegetables as a separate category), potatoes, nuts and seeds, and soy, revealed no differences. Additionally, dietary factors that influence the absorption of dietary oxalate from the gastrointestinal tract (e.g., calcium and magnesium) were not different between groups, further supporting a role for differences in intestinal microbiome composition in affecting the absorption of components relevant to stone formation.

The intestinal microbiome is known to play a significant role in maintaining overall health, and its dysbiosis has been linked to numerous disease states including USD. For calcium oxalate USD specifically, the primary species of interest has been O. formigenes, although other facultative oxalate-degrading species have been the subject of research as well. Several factors suggest that while O. formigenes and other oxalate-degrading bacteria are indeed associated with oxalate metabolism in the gut, they are not solely responsible for the function nor are they sufficient to inhibit calcium oxalate USD. First, while a majority of patients are found not to be colonized by this strict oxalate-degrading bacterium, the same is true of non-stone forming individuals. Besides, many patients with calcium oxalate USD are colonized by O. formigenes. Second, while probiotics containing O. formigenes or a mix of facultative oxalate-degrading bacteria do reduce urinary oxalate excretion, their effect, if at all, is typically ephemeral. In contrast, when given whole fecal transplants from N. albigula, mammals whose gastrointestinal tracts harbor highly efficient bacterial oxalate degrading networks, Sprague-Dawley rats exhibit a marked and persistent decrease in urinary oxalate. Third, the intestinal microbiome is a highly symbiotic environment consisting of complex and integrated functional microbial networks.

In conclusion, the results suggest that healthy oxalate homeostasis in the gastrointestinal tract may not be attributed to the action of O. formigenes alone, but may rather involve a collaborative effort between numerous bacterial species, including Ruminococcus and Oscillospira. This would be consistent with the highly symbiotic environment of the intestinal microbiome, an example of which is the co-colonization in the samples of methanogens species with O. formigenes. Furthermore, the data suggest a potential role for the loss of members of the oxalate microbiome in increasing the risk for the formation of non-oxalate stones, which is consistent with the highly symbiotic nature of the intestinal microbiome.

### Materials and Methods

Included subjects had no antibiotic exposure within 30-days prior to providing the sample and based on detailed medical history obtained at the time of enrolment had no interventions/treatments that impacted intestinal microbiome composition. The total number of daily bowel movements per patient was not determined. Patients had at least 1 recurrence of their stones and no family history of primary hyperoxaluria, while controls lived in the same household as patients and had no personal or family history of USD. Subject demographics are given in Table 3.

Power analysis based upon results from previous studies of microbiota composition revealed that the sample size gave a 65% power of detecting a difference in community composition and differential abundance.

Dietary intake of both cases and controls was assessed using the Canadian version of the National Institutes of Health Diet History Questionnaire (C-DHQ-I). This instrument is a publicly available food frequency questionnaire consisting of 134 food items — based on the national dietary data from the National Health and Nutrition Examination Surveys from 2001-2002, 2003-2004, and 2005-2006 — and 11 dietary supplement questions. The C-DHQ-I queries subjects about intake in the past year, assessing for habitual intake, and includes questions about portion size. The paper-and-pencil version of the questionnaire was provided to each subject prior to his/her fecal sample. Data from the C-DHQ-I were analyzed with the Diet*Calc software developed by the National Cancer Institute specifically for this instrument. Nutrient and food group intake estimates were calculated.

Fecal samples were collected by study participants into provided containers on the morning of sample delivery. Upon arrival at a facility (within 4 hours of defecation), fecal samples were stored at 4° C. before being aliquoted into microfuge tubes and subsequent storage at -80° C. until DNA extraction.

Fecal DNA was extracted and purified using the QIAamp DNA Stool Mini Kit (51504, Qiagen) according to the manufacturer’s instructions with modifications to cell lysis buffer (4% (w/v) SDS, 500 mM NaCl, 50 mM EDTA, 50 mM Tris pH 8.0) and use of extra glass lysis beads (0.3 g of 0.1 mm beads and 0.1 g of 0.5 mm beads) for a more thorough lysis of Firmicutes bacteria.

A 16 S rRNA library was prepared from the fecal DNA based on a protocol by Kozich et al., Appl Environ Microbiol, 79:5112-20 (2013)). The extracted DNA was amplified using the Phusion Hot Start II DNA Polymerase (2U/ul) kit (F549S, Thermo Fisher Scientific) in 50 ul reactions according to the manufacturer’s instructions with the following modifications to the PCR cycle; initial denaturation at 98° C. for 2 minutes, 30 cycles of 98° C. for 20 s; 55° C. for 15 s; and 72° C. for 30 s extensions; followed by a final extension at 72° C. for 10 minutes and holding at 4° C. To validate PCR success, a random subset of PCR products was analyzed for visible bands on gel electrophoresis. The PCR products were cleaned using Agencourt Ampure XP beads (A63880, Beckman Coulter) using a 0.8:1 bead to sample ratio. The cleaned PCR products were normalized using the SequalPrep Normalization Plate kit (A1051001, Invitrogen) to a concentration of 1-2 ng/ul. 5 ul from each normalized sample was pooled into a single library and further concentrated using the DNA Clean & Concentrator-5 kit (D4013, Zymo Research). The pooled library was analyzed on the Agilent Bioanalyzer using the High Sensitivity DS DNA assay (5067-4626, Agilent) to determine approximate library fragment size and to verify library integrity. The QIAquick Gel Extraction kit (28704, Qiagen) was used to extract properly-sequenced 16 S rRNA amplicons in the pooled library and exclude unintended amplicons. The concentration of the final pooled library was determined using the KAPA Library Quantification Kit for Illumina (KK4824, Kapa Biosystems). The library was then diluted to 4nM and denatured into single strands using 0.2 N NaOH. The final library loading concentration was 8 pM with an additional 20% PhiX (FC-110-3001, Illumina) spike-in for sequencing quality control. The 16 S rRNA pooled library was then sequenced on an Ilumina MiSeq platform.

Unless otherwise noted, all analyses were conducted in QIIME 59. Raw sequencing data were demultiplexed with default parameters, and operational taxonomic units (OTUs) were assigned de novo at a 97% homology cutoff, using UCLUST 60. Data were filtered to remove mitochondria, chloroplasts, and sequences with <10 representations across the entire dataset. Filtered data were used to summarize taxa, determine which OTUs differed between groups, and quantify bacteria that co-occur with Oxalobacter sp. (discussed below).

Prior to comparative analyses, data were normalized with the DESeq2 algorithm, which executes a negative binomial Wald test while maintaining rare taxa. For alpha-diversity, Margalef’s species richness, evenness, the Shannon index, and phylogenetic diversity were calculated. Additionally, beta-diversity was calculated using both unweighted (membership) and weighted (structure) UniFrac analyses, followed by a post-hoc 2-way PERMANOVA analysis against USD-status and gender as factors 63. The differential abundance of OTUs between USD and healthy groups was calculated using a Wald test, which determines significance by the log2 fold change of the normalized OTU abundance, divided by its standard error. Resulting p-values were adjusted to account for false discoveries.

To compare differences between groups in the microbial network associated with oxalate metabolism, several metrics were calculated. First, the normalized relative abundance of Oxalobacter sp. was compared with a t-test. Second, the proportion of individuals colonized by Oxalobacter sp. in each group was compared with a relative risk analysis in R statistical software. Finally, with the assumption that Oxalobacter is a central component of a broader microbial network associated with oxalate metabolism, the bacteria that exhibited a relative abundance that significantly and positively correlated with Oxalobacter were quantified, as assessed by false discovery rate corrected Pearson correlations. The resulting list was used to quantify the co-occurrence network in the healthy and USD groups. Co-occurrence was determined using the SparCC algorithm, by group, as previously described.

Sequence reads are available at the Sequence Read Archive under Accession # SRP140933.

### Example 3: Effect of Antibiotics on the Gut Microbiota

The objective of the study was to determine the nature and location of dysbiosis associated with USD. Microbiome analysis from the gastrointestinal and urinary tracts was conducted, along with a metabolomic analysis of the urinary metabolome, from subjects with an active episode of USD or no history of the disease. Higher rates of antibiotic use among USD patients along with integrated microbiome and metabolomic results support the hypothesis that USD is associated with an antibiotic-driven shift in the microbiome from one that protects against USD to one that promotes the disease. Specifically, the study implicates urinary tract Lactobacillus and Enterobacteriaceae in protective and pathogenic roles for USD, respectively, which conventional, culture-based methods of bacterial analysis from urine and kidney stones would not necessarily detect. Results suggest that antibiotics produce a long-term shift in the microbiome that may increase the risk for USD, with the urinary tract microbiome holding more relevance for USD than the gut microbiome.

Dysbiosis, the contribution of the microbiome to disease processes, can come in one of three different forms. First, a shift in the microbiome can lead to the emergence of bacteria and functions that cause disease, herein referred to as gain of function dysbiosis. Gain of function dysbiosis results from the overgrowth of pathogens that lead to diseases such as cholera, strep throat, or E. coli infection. Second, a shift in the microbiome can lead to the loss of bacteria and functions that protect health, herein referred to as loss of function dysbiosis. Loss of function dysbiosis is inherently more difficult to attribute to a disease process as it is by definition, the absence of specific bacteria from a complex microbiome that causes a disease rather than their presence. Regardless, loss of function dysbiosis is increasingly being recognized as an important contributor to many diseases including inflammatory bowel disease (IBD), obesity, cardiovascular disease, asthma, and others. Finally, a combination of loss and gain of function dysbiosis may contribute to or be required for some disease processes. Such is the case with recurrent Clostridium difficile infection, in which repeated antibiotic use leads to the depletion of the commensal microbiota, which allows for the proliferation of pathogenic C. difficile.

The objective of the current study was to take a multi-specimen, multi-omic approach to specifically determine 1) if calcium-based and uric acid kidney stones are significantly associated with microbial dysbiosis; 2) the site of microbial activity that is most important for USD; and 3) factors that impact the microbiome in a way that facilitates the onset of USD. The goal of the work was to provide a solid foundation to translate results obtained from microbiome studies associated with USD into effective, persistent, and personalized bacteriotherapies to prevent USD.

### Results

### Clinical Cohort of Participants

A total of 67 individuals were recruited for the current study, with 43 subjects that had no history of USD, and 24 subjects with an active episode of USD. The USD patients had stones composed of calcium oxalate, calcium phosphate, uric acid, or a mixture of components. Patients with a history of struvite stones were not recruited since these stones are known to be derived from pathogenic Enterobacteriaceae bacteria. Consistent with previous reports, the healthy and USD cohorts differed significantly by age, diabetes, 12-month antibiotic use, and family history of USD (Table 8) and is thus an adequate representation of the USD population.

Table 8

Patient metadata

| Metric | Healthy | USD | P-value | Statistic |
|---|---|---|---|---|
| No. enrolled | 43 | 24 | NA | NA |
| % Antibiotics used in past 12 months | 39.53% | 75% | 0.01 | Relative risk, Fisher Exact Test |
| Age | 35.47 +/-1.73 | 51.54 +/-2.53 | <0.001 | Student’s t-test |
| % Prior USD | 0% | 75% | <0.001 | Relative risk, Fisher Exact Test |
| % Diabetic | 2.33% | 25% | 0.007 | Relative risk, Fisher Exact Test |
| % CaOx | | 43% | | |
| % CaOx + CaPhos | | 22% | | |
| % CaOx + Uric acid | | 4% | | |
| % CaPhos | | 13% | | |
| % Uric acid | | 17% | | |
| % Family History of USD | 26% | 41% | 0.016 | Relative risk, Fisher Exact Test |
| % Antibiotics used in past 30 days | 0% | 4.17% | 0.35 | Relative risk, Fisher Exact Test |
| Height (cm) | 171.54 +/-1.53 | 171.66 +/-1.7 | 0.5 | Student’s t-test |
| Weight (kg) | 76 +/- 3.26 | 81 +/- 6.55 | 0.59 | Student’s t-test |
| % Female | 60% | 58% | 0.785 | Relative risk, Fisher Exact Test |
| % Gastrointestinal Illness | 6.98% | 8.33% | 1 | Relative risk, Fisher Exact Test |

P-values for significantly different metrics are bolded. * Values expressed as mean +/ standard error.

### 16S rRNA Sequencing and Untargeted Metabolomics

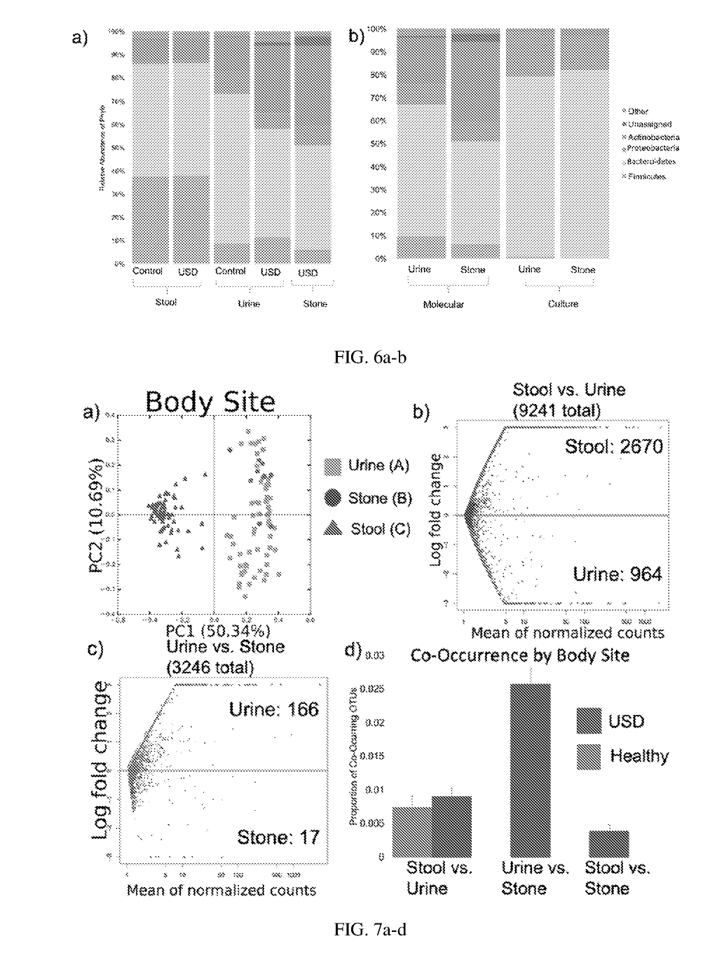

A total of 199 DNA samples from stool, urine, kidney stones, and cultures generated from urine and kidney stones, were subjected to high-throughput sequencing of the V4 region of the 16S rRNA gene. Sequencing resulted in 12,020 ,020 high quality sequences, used for downstream analyses. A moderate abundance-based operational taxonomic unit (OTU) filtering strategy was employed that balances removing spurious OTUs with maintaining rare OTUs, as done previously. With this strategy, a total of 7,376 (1432 +/- 65 per sample), 3,308 (452 +/- 24 per sample), and 473 (341 +/- 74 per sample) unique operational taxonomic units (OTUs) were defined in the stool, urine, and kidney stone samples, respectively, when DNA was extracted directly from samples. From urine and stone cultures, 2,068 (393 +/- 37 per sample) and 635 (137 +/- 23 per sample) OTUs were defined. All samples were represented by a high abundance of sequence reads (>3,000 for urine, stones, or cultures; >10,000 for stool). Taxonomic assignment at the phylum level ranged from 96% (stones) to >99% (stool; FIG. 6), while genus level assignment ranged from 68% (stones) to 86% (urine).

For untargeted urinary metabolomics, 31 samples from healthy individuals and 18 samples from USD patients were analyzed. Analysis resulted in 13,348 high quality and unique spectral features. Of these, 2,110 were assigned putative identification, either with mass spectrometry alone or with tandem mass spectrometry.

### Analysis of the Microbiome by Specimen-Type, Technique, and USD Status

The composition of the microbiota from the three specimen-types (urine, stool, and kidney stone) were unique as assessed by beta-diversity (FIG. 7a, Table 9). There was an average of ∼0.8% co-occurrence of OTUs between the urine and stool with 39% of the OTUs exhibiting significant differential abundance (FIGS. 7b,d). This compares to an average of 2.5% of OTUs co-occurring in both urine and stone with only 6% of the OTUs exhibiting significant differential abundance (FIGS. 7c,d).

Table 9

List of significant pairwise comparisons of weighted UniFrac beta-diversity

| Comparison | Adonis p-value |
|---|---|
| Stool vs. Urine | 0.008 |
| Stool vs. Stone | 0.008 |
| Urine vs. Stone | 0.008 |
| Urine by 12 m Antibiotic use | 0.022 |
| Urine by Technique | 0.008 |
| Stone by Technique | 0.048 |
| Urine by USD-status | 0.009 |
| Urine by Family history of USD | 0.042 |
| Urine by Sex | 0.008 |

Stool/Urine/Stone = Specimen that the microbiome data originated from; USD-status = Healthy vs. USD; Technique = Molecular vs. Culture. P-values were corrected for multiple comparisons using a Holm’s correction.

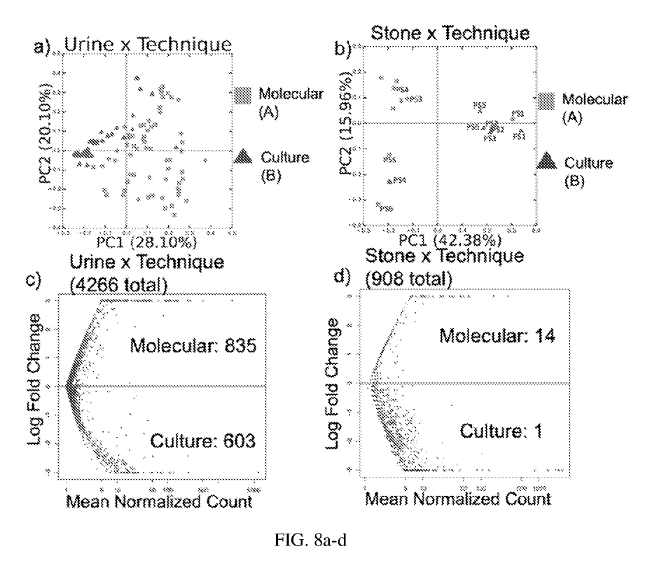

To examine the effect of culturing on the bacteria detected by sequencing, 16S rRNA microbial inventories generated from urine and stone samples where DNA had been extracted directly from samples, or after they had been cultured on blood agar, were compared (FIG. 8). In blood agar, bacteria were successfully cultured from 30 out of the 43 urine samples of healthy subjects and 19 out of the 24 USD subjects. Additionally, bacteria were successfully cultured in seven of the 10 stone samples. Species detected from urine and stones were dependent on whether bacteria were cultured prior to DNA extraction, with a greater diversity of OTUs detected when DNA was directly extracted from samples for urine but not stone samples (FIGS. 6 and 8).

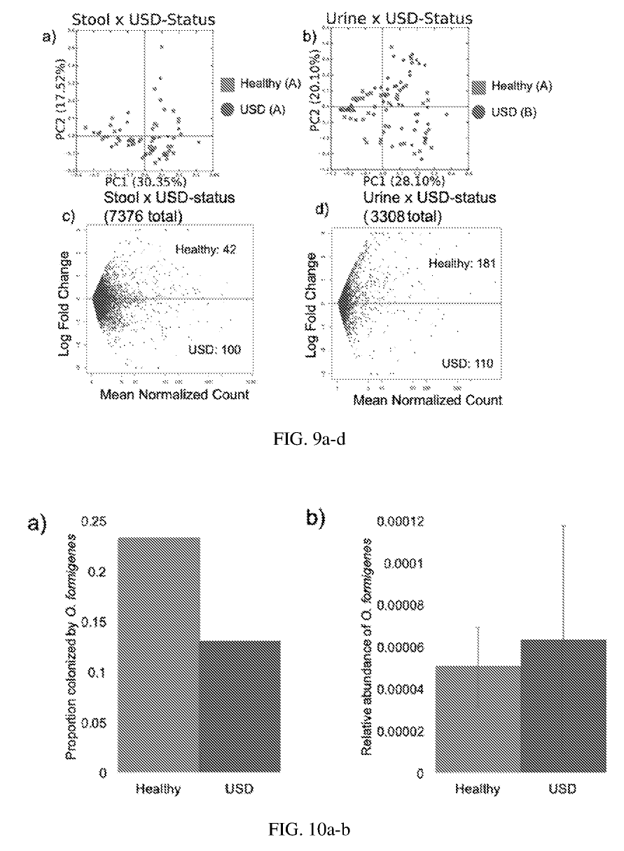

For the stool microbiome, there was no difference in beta-diversity of the microbiota between the healthy and USD cohorts (FIG. 9a). However, there was a USD-dependent difference in the composition of the urinary microbiome (FIG. 9b). Furthermore, differential OTU abundance analysis revealed that only 1.9% of the OTUs were significantly different in the stool microbiome between the healthy and USD cohorts with 2.4 fold more OTUs enriched in the USD cohort compared to healthy cohort (FIG. 9c, Table 10). For the urinary enriched in the healthy cohort compared to the USD cohort (FIG. 9d, Table 10). The taxa that differentiated the healthy cohort from the USD cohort most were the Lachnospiraceae in the stool of the USD cohort, Lactobacillus in the urine of the healthy cohort, and the Enterobacteriaceae in the urine of the USD covvhort (Table 10).

Table 10

OTUs that were differentially abundant between the healthy and USD cohorts. Data were assessed with a negative binomial Wald test

| Specimen | Group | Lowest Assigned Taxonomy | Taxonomic Level | No. of OTUs | Log2 Fold Change | FDR |
|---|---|---|---|---|---|---|
| Stool | Healthy | Clostridiales | Order | 7 | 1.56-3.2 | <0.001-0.036 |
| Stool | Healthy | Ruminococcaceae | Family | 5 | 1.49-2.17 | 0.007-0.03 |
| Stool | Healthy | Bacteroides | Genus | 3 | 2.47-3.23 | <0.001-0.007 |
| Stool | Healthy | Rikenellaceae | Family | 3 | 1.64-2.05 | 0.037 |
| Stool | Healthy | Bifidobacterium | Genus | 2 | 1.51-1.87 | 0.008-0.009 |
| Stool | Healthy | Coprococcus | Genus | 2 | 2.29-2.51 | 0.007 |
| Stool | Healthy | Pseudomonas | Genus | 2 | 1.64-7.29 | <0.001-0.045 |
| Stool | Healthy | Varibaculum | Genus | 2 | 1.55-1.89 | 0.027-0.036 |
| Stool | Healthy | Akkermansia | Genus | 1 | 2.02 | 0.033 |
| Stool | Healthy | Barnesiellaceae | Family | 1 | 1.78 | 0.019 |
| Stool | Healthy | Clostridia | Class | 1 | 1.68 | 0.017 |
| Stool | Healthy | Clostridiaceae | Family | 1 | 2.4 | <0.001 |
| Stool | Healthy | Clostridium | Genus | 1 | 1.66 | 0.045 |
| Stool | Healthy | Corynebacterium | Genus | 1 | 3.79 | 0.04 |
| Stool | Healthy | Eubacterium | Genus | 1 | 2.47 | <0.001 |
| Stool | Healthy | Lachnospiraceae | Family | 1 | 1.42 | 0.007 |
| Stool | Healthy | Paraprevotella | Genus | 1 | 2.68 | <0.001 |
| Stool | Healthy | Prevotella | Genus | 1 | 1.66 | 0.033 |
| Stool | Healthy | RF32 | Order | 1 | 2.64 | 0.002 |
| Stool | Healthy | Ruminococcus | Genus | 1 | 2.05 | 0.018 |
| Stool | Healthy | Turicibacter | Genus | 1 | 2.06 | 0.005 |
| Stool | Healthy | YS2 | Order | 1 | 1.51 | 0.048 |
| Stool | USD | Lachnospiraceae | Family | 33 | 1.09-2.02 | <0.001-0.049 |
| Stool | USD | Bacteroides | Genus | 20 | 1.11-1.67 | 0.012-0.048 |
| Stool | USD | Enterobacteriaceae | Family | 5 | 1.28-1.32 | <0.001 |
| Stool | USD | Megasphaera | Genus | 5 | 1.16-3.67 | <0.001-0.047 |
| Stool | USD | Ruminococcaceae | Family | 4 | 1.38-2.18 | <0.001-0.047 |
| Stool | USD | Dorea | Genus | 3 | 1.22-3.77 | <0.001-0.036 |
| Stool | USD | Clostridiales | Order | 2 | 1.37-1.77 | 0.003-0.018 |
| Stool | USD | Coprococcus | Genus | 2 | 1.8-1.97 | 0.009-0.016 |
| Stool | USD | Rikenellaceae | Family | 2 | 1.66-7.64 | 0.007-0.014 |
| Stool | USD | Ruminococcus | Genus | 2 | 1.37-1.77 | 0.036-0.042 |
| Stool | USD | Actinomyces | Genus | 1 | 1.22 | 0.026 |
| Stool | USD | Atopobium | Genus | 1 | 1.36 | 0.048 |
| Stool | USD | Blautia | Genus | 1 | 1.89 | 0.03 |
| Stool | USD | Christensenellaceae | Family | 1 | 1.06 | 0.048 |
| Stool | USD | Citrobacter | Genus | 1 | 3.65 | <0.001 |
| Stool | USD | Comamonadaceae | Family | 1 | 2.61 | <0.001 |
| Stool | USD | Coriobacteriaceae | Family | 1 | 1.94 | <0.001 |
| Stool | USD | Eubacterium | Genus | 1 | 1.86 | 0.009 |
| Stool | USD | Fusobacterium | Genus | 1 | 1.85 | <0.001 |
| Stool | USD | ML615J-28 | Order | 1 | 1.11 | 0.048 |
| Stool | USD | Odoribacter | Genus | 1 | 1.81 | 0.001 |
| Stool | USD | Oscillospira | Genus | 1 | 2.82 | <0.001 |
| Stool | USD | Parabacteroides | Genus | 1 | 1.96 | 0.01 |
| Stool | USD | Paraprevotella | Genus | 1 | 3.45 | <0.001 |
| Stool | USD | Peptococcaceae | Family | 1 | 2.04 | 0.003 |
| Stool | USD | Peptostreptococcaceae | Family | 1 | 1.04 | 0.049 |
| Stool | USD | RF39 | Order | 1 | 2.69 | <0.001 |
| Stool | USD | Roseburia | Genus | 1 | 1.37 | 0.008 |
| Stool | USD | Shuttleworthia | Genus | 1 | 1.76 | 0.021 |
| Stool | USD | Sneathia | Genus | 1 | 1.91 | <0.001 |
| Urine | Healthy | Lactobacillus | Genus | 100 | 1.03-7.22 | <0.001-0.048 |
| Urine | Healthy | Prevotella | Genus | 14 | 1.15-4.41 | <0.001-0.034 |
| Urine | Healthy | Corynebacterium | Genus | 6 | 1.17-2.67 | <0.001-0.049 |
| Urine | Healthy | Finegoldia | Genus | 6 | 1.1-2.53 | 0.002-0.044 |
| Urine | Healthy | WAL 1855D | Genus | 5 | 1.22-1.52 | 0.015-0.049 |
| Urine | Healthy | Porphyromonas | Genus | 4 | 1.88-2.67 | <0.001-0.015 |
| Urine | Healthy | Anaerococcus | Genus | 3 | 1.38-1.86 | 0.002-0.003 |
| Urine | Healthy | Campylobacter | Genus | 2 | 1.45-2.18 | <0.001-0.015 |
| Urine | Health | Clostridiales | Order | 2 | 1.46-1.49 | 0.014-0.048 |
| Urine | Healthy | Dialister | Genus | 2 | 1.54-2.11 | 0.009-0.038 |
| Urine | Healthy | Megasphaera | Genus | 2 | 2.63 | <0.001 |
| Urine | Healthy | Peptoniphilus | Genus | 2 | 1.35-1.76 | 0.006-0.024 |
| Urine | Healthy | Peptostreptococcus | Genus | 2 | 1.16-3.02 | <0.001-0.024 |
| Urine | Healthy | Veillonella | Genus | 2 | 1.84-3.27 | <0.001-0.004 |
| Urine | Healthy | Acinetobacter | Genus | 1 | 2.93 | 0.002 |
| Urine | Healthy | Actinomyces | Genus | 1 | 2.93 | <0.001 |
| Urine | Healthy | Actinomycetales | Order | 1 | 2.21 | <0.001 |
| Urine | Healthy | Atopobium | Genus | 1 | 1.81 | 0.004 |
| Urine | Healthy | Bacillus | Genus | 1 | 1.99 | <0.001 |
| Urine | Healthy | Bifidobacterium | Genus | 1 | 4.53 | <0.001 |
| Urine | Healthy | Brevibacterium | Genus | 1 | 1.65 | 0.007 |
| Urine | Healthy | Clostridium | Genus | 1 | 1.33 | 0.047 |
| Urine | Healthy | Comamonadaceae | Family | 1 | 1.6 | 0.035 |
| Urine | Healthy | Coriobacteriaceae | Family | 1 | 2.51 | <0.001 |
| Urine | Healthy | Elizabethkingia | Genus | 1 | 1.66 | 0.004 |
| Urine | Healthy | Gallicola | Genus | 1 | 1.22 | 0.044 |
| Urine | Healthy | Granulicatella | Genus | 1 | 2.99 | <0.001 |
| Urine | Healthy | Haemophilus | Genus | 1 | 3.32 | <0.001 |
| Urine | Healthy | Mobiluncus | Genus | 1 | 3.2 | <0.001 |
| Urine | Healthy | Neisseria | Genus | 1 | 1.32 | 0.021 |
| Urine | Healthy | Neisseriaceae | Family | 1 | 1.32-1.69 | 0.005 |
| Urine | Healthy | Peptococcus | Genus | 1 | 1.3 | 0.034 |
| Urine | Healthy | ph2 | Genus | 1 | 1.75 | 0.009 |
| Urine | Healthy | Rhodocyclaceae | Family | 1 | 1.57 | 0.009 |
| Urine | Healthy | Rothia | Genus | 1 | 1.34 | 0.025 |
| Urine | Healthy | Ruminococcaceae | Family | 1 | 1.27 | 0.025 |
| Urine | Healthy | Sphingobium | Genus | 1 | 2.29 | 0.001 |
| Urine | Healthy | Sphingomonas | Genus | 1 | 1.2 | 0.039 |
| Urine | Healthy | Staphylococcus | Genus | 1 | 1.95 | 0.015 |
| Urine | Healthy | Streptococcus | Genus | 1 | 1.9 | 0.003 |
| Urine | Healthy | Streptomyces | Genus | 1 | 1.32 | 0.039 |
| Urine | Healthy | Tissierellaceae | Family | 1 | 1.01 | 0.012-0.049 |
| Urine | Healthy | Weeksellaceae | Family | 1 | 1.46-1.66 | 0.024 |
| Urine | USD | Enterobacteriaceae | Family | 31 | 0.94-2.36 | <0.001-0.049 |
| Urine | USD | Veillonella | Genus | 20 | 0.87-1.47 | 0.003-0.048 |
| Urine | USD | Enterococcus | Genus | 7 | 0.91-2.21 | <0.001-0.044 |
| Urine | USD | Flavohacterium | Genus | 5 | 0.97-5.37 | <0.001-0.049 |
| Urine | USD | Meashaera | Genus | 3 | 1.2-. | <0.001-0.023 |
| Urine | USD | Pseudomonas | Genus | 3 | 1.14-3.83 | <0.001-0.023 |
| Urine | USD | Serratia | Genus | 3 | 0.94-1.15 | 0.016-0.045 |
| Urine | USD | Delftia | Genus | 2 | 1.58-3.28 | <0.001-0.003 |
| Urine | USD | Enterococcaceae | Family | 2 | 0.91-2.21 | 0.003-0.039 |
| Urine | USD | Prevotella | Genus | 2 | 1.61-2.32 | 0.002-0.007 |
| Urine | USD | Acetobacter | Genus | 1 | 1.09 | 0.03 |
| Urine | USD | Acinetobacter | Genus | 1 | 1.11 | 0.029 |
| Urine | USD | Agrobacterium | Genus | 1 | 0.96 | 0.033 |
| Urine | USD | Allobaculum | Genus | 1 | 1.59 | 0.002 |
| Urine | USD | Bacillaceae | Family | 1 | 1.09-1.91 | 0.011 |
| Urine | USD | Bifidobacteriaceae | Family | 1 | 3.33 | <0.001 |
| Urine | USD | Clostridiales | Order | 1 | 1.34 | 0.01 |
| Urine | USD | Clostridium | Genus | 1 | 1.18 | 0.05 |
| Urine | USD | Coriobacteriaceae | Family | 1 | 1.59 | <0.001 |
| Urine | USD | Corynebacterium | Genus | 1 | 1.57 | 0.002 |
| Urine | USD | Cryocola | Genus | 1 | 2.21 | <0.001 |
| Urine | USD | Fluviicola | Genus | 1 | 1.24 | 0.008 |
| Urine | USD | Fusobacteriales | Order | 1 | 1 | 0.022 |
| Urine | USD | Geobacillus | Genus | 1 | 2.13 | <0.001 |
| Urine | USD | Lactobacillus | Genus | 1 | 1.07 | 0.039 |
| Urine | USD | Microbacteriaceae | Family | 1 | 1.07-1.61 | 0.002 |
| Urine | USD | Mycoplana | Genus | 1 | 0.94 | 0.049 |
| Urine | USD | Neisseriaceae | Family | 1 | 1.11 | 0.034 |
| Urine | USD | Oxalobacteraceae | Family | 1 | 2.29 | <0.001 |
| Urine | USD | Paracoccus | Genus | 1 | 1.14 | 0.024 |
| Urine | USD | Peptoniphilus | Genus | 1 | 1.01 | 0.036 |
| Urine | USD | Porphyromonas | Genus | 1 | 1.61 | 0.002 |
| Urine | USD | Rhodanobacter | Genus | 1 | 2.02 | 0.002 |
| Urine | USD | Rhodobacter | Genus | 1 | 1.17 | 0.032 |
| Urine | USD | S24-7 | Family | 1 | 1.08 | 0.031 |
| Urine | USD | Sediminibacterium | Genus | 1 | 1.47 | 0.004 |
| Urine | USD | Sphingomonas | Genus | 1 | 0.97 | 0.05 |
| Urine | USD | Staphylococcus | Genus | 1 | 1.41 | 0.01 |
| Urine | USD | Stenotrophomonas | Genus | 1 | 1.32 | 0.015 |
| Urine | USD | Unassigned | NA | 1 | 1.57 | 0.038 |
| Urine | USD | Xanthomonadaceae | Family | 1 | 1.32-2.02 | 0.009 |

The composition of the urinary tract microbiome also differed by 12-month antibiotic use, sex, and family history of USD (Table 9). However, urinary tract microbiota composition did not differ significantly by age, diabetic-status, diet, 30-day antibiotic use, height, weight, whether the patient had gout or hypertension (data not shown).

In our dataset, colonization by O. formigenes was 23% of healthy individuals and 13% of USD patients, with no significant difference between groups as determined by a relative risk analysis, followed by a post-hoc Fisher’s exact test (FIG. 10a). The relative abundance of O. formigenes was also not significantly different by group, as determined by a t-test (FIG. 10b). Furthermore, neither 30-day nor 12-month antibiotic use had a significant correlation to O. formigenes colonization.

### Urinary Metabolomics by USD-Status

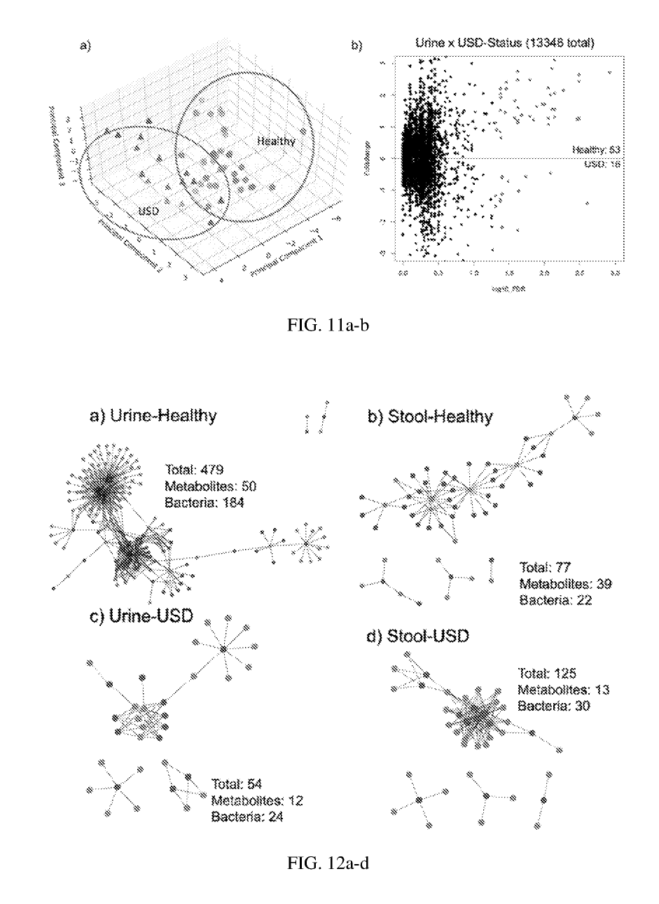

Overall, the urinary metabolome clustered by USD-status, based on principal coordinates analysis (PCoA) of the log-transformed, creatinine-normalized, metabolite concentrations (FIG. 11a). When examining the differential concentration of individual metabolites by USD-status, 53 were enriched in the healthy group, with 16 enriched in the USD group, representing a 3.3 fold higher number of metabolites enriched in the healthy cohort compared to the USD cohort (FIG. 11b). Combined, these metabolites made up 0.05% of the total number of metabolites defined in the dataset (Table 11).

Table 11

All urinary metabolites that were significantly different between the healthy and USD cohorts. Significance was determined by a FDR-corrected Welch’s t-test of metabolite concentrations normalized to creatinine. m/z: mass to charge ratio

| Group | m/z | ID | Fold Change (USD/Healthy) | FDR |
|---|---|---|---|---|
| Healthy | 451.14095 | Unknown | 0.14 | 0.001 |
| Healthy | 365.14278 | Unknown | 0.27 | 0.001 |
| Healthy | 367.15834 | DHEA | 0.10 | 0.002 |
| USD | 363.92954 | Unknown | 1.42 | 0.002 |
| Healthy | 447.23893 | LysoPC(10:0) | 0.27 | 0.003 |
| Healthy | 368.16185 | Unknown | 0.09 | 0.003 |
| Healthy | 384.15701 | Unknown | 0.18 | 0.003 |
| Healthy | 386.17266 | Unknown | 0.29 | 0.003 |
| Healthy | 445.19031 | Unknown | 0.44 | 0.003 |
| Healthy | 601.28833 | Unknown | 0.45 | 0.003 |
| Healthy | 600.28486 | Unknown | 0.45 | 0.003 |
| USD | 114.12774 | Unknown | 3.79 | 0.005 |
| Healthy | 383.15353 | 3b,16a-Dihydroxyandrostenone sulfate | 0.19 | 0.005 |
| Healthy | 495.29639 | Pregnanediol-3-glucuronide, 3-alpha,20-alpha-dihydroxy-5-beta-pregnane 3-glucuronide | 0.32 | 0.005 |
| Healthy | 453.15658 | Unknown | 0.33 | 0.007 |
| Healthy | 565.17436 | Unknown | 0.39 | 0.007 |
| Healthy | 602.29251 | Unknown | 0.43 | 0.007 |
| Healthy | 130.06218 | Creatine, Beta-Guanidinopropionic acid | 0.24 | 0.007 |
| Healthy | 413.20054 | Unknown | 0.26 | 0.008 |
| Healthy | 596.21071 | Unknown | 0.39 | 0.008 |
| Healthy | 385.16919 | Unknown | 0.33 | 0.008 |
| USD | 409.07532 | Unknown | 1.47 | 0.008 |
| Healthy | 496.29976 | Unknown | 0.31 | 0.009 |
| Healthy | 466.25256 | Unknown | 0.44 | 0.009 |
| Healthy | 406.07565 | Unknown | 0.48 | 0.009 |
| Healthy | 509.27541 | Unknown | 0.37 | 0.010 |
| Healthy | 510.27881 | Unknown | 0.37 | 0.011 |
| Healthy | 468.25789 | Unknown | 0.38 | 0.013 |
| Healthy | 465.24916 | Androsterone glucuronide | 0.45 | 0.014 |
| Healthy | 437.16169 | Unknown | 0.29 | 0.016 |
| Healthy | 467.25548 | Unknown | 0.43 | 0.016 |
| USD | 642.15525 | Unknown | 1.58 | 0.018 |
| Healthy | 463.23385 | Testosterone glucuronide, Dehydroisoandrosterone 3-glucuronide, Dehydroepiandrosterone 3-glucuronide | 0.42 | 0.021 |
| USD | 265.97993 | Unknown | 2.03 | 0.023 |
| Healthy | 383.15359 | 3b,16a-Dihydroxyandrostenone sulfate | 0.38 | 0.024 |
| Healthy | 299.07732 | Unknown | 0.23 | 0.024 |
| Healthy | 533.23669 | Unknown | 0.47 | 0.024 |
| Healthy | 401.16410 | Unknown | 0.27 | 0.024 |
| Healthy | 448.24231 | Unknown | 0.29 | 0.024 |
| Healthy | 595.20746 | Unknown | 0.46 | 0.024 |
| USD | 197.80804 | Unknown | 3.27 | 0.024 |
| Healthy | 215.99732 | Unknown | 0.35 | 0.026 |
| Healthy | 663.19475 | Unknown | 0.44 | 0.026 |
| Healthy | 412.18834 | Unknown | 0.06 | 0.032 |
| Healthy | 468.13091 | Unknown | 0.05 | 0.033 |
| Healthy | 452.14441 | Unknown | 0.05 | 0.033 |
| USD | 325.09299 | Unknown | 1.72 | 0.033 |
| Healthy | 563.28385 | Unknown | 0.29 | 0.034 |
| Healthy | 370.17756 | Unknown | 0.31 | 0.034 |
| Healthy | 463.23387 | Testosterone glucuronide, Dehydroisoandrosterone 3-glucuronide, Dehydroepiandrosterone 3-glucuronide | 0.41 | 0.036 |
| Healthy | 270.50875 | Unknown | 0.62 | 0.040 |
| USD | 644.29572 | Unknown | 1.13 | 0.040 |
| USD | 295.22436 | (R)-3-Hydroxy-hexadecanoic acid | 1.60 | 0.040 |
| USD | 171.06517 | 3,4-Dihydroxyphenylglycol | 3.17 | 0.040 |
| USD | 299.06344 | Unknown | 1.58 | 0.040 |
| Healthy | 497.30269 | Unknown | 0.34 | 0.041 |
| Healthy | 313.08083 | Unknown | 0.61 | 0.041 |
| Healthy | 369.17406 | Androsterone sulfate, 5a-Dihydrotestosterone sulfate, Etiocholanolone sulfate | 0.33 | 0.042 |
| Healthy | 147.06633 | Lactose, Maltose | 0.54 | 0.042 |
| USD | 192.06672 | 2-Methylhippuric acid | 2.36 | 0.042 |
| Healthy | 429.19526 | Unknown | 0.31 | 0.042 |
| Healthy | 387.18489 | Unknown | 0.40 | 0.042 |
| USD | 276.09121 | Unknown | 2.30 | 0.042 |
| USD | 114.18528 | Unknown | 12.28 | 0.044 |
| Healthy | 452.13616 | Unknown | 0.02 | 0.046 |
| Healthy | 115.00367 | Fumaric acid, Maleic acid | 0.53 | 0.047 |
| USD | 327.11745 | Unknown | 2.51 | 0.049 |
| Healthy | 245.13951 | Hexanoylglycine | 0.47 | 0.049 |
| USD | 259.12996 | L-gamma-glutamyl-L-isoleucine, L-gamma-glutamyl-L-leucine | 2.18 | 0.049 |

### Functional Microbial Networks by Specimen-Type and USD-Status

Finally, the microbe-metabolite interactions that most differentiated the healthy population from the USD population were determined. To do so, the microbiome data was integrated with the metabolomic data by conducting pairwise Pearson correlations between the DESeq2-normalized OTU counts that were enriched in either the healthy or USD groups for either the fecal or urinary microbiome and the creatinine-normalized urine metabolite concentrations that were enriched in either the healthy or USD groups. This analysis revealed that what differentiated the healthy cohort from the USD cohort was primarily the loss of Lactobacillus from the urinary tract of the healthy population, associated with three currently unknown metabolites (FIG. 12, Table 12).

Table 12

List significant microbe-metabolite interactions by group and sample-type

| Metabolite (m/z) | OTU | No. of OTUs /metabolite | Group |
|---|---|---|---|
| Unknown (-387.184893709281) | Lactobacillus | 63 | Healthy-Urine |
| Unknown (-406.075648313136) | Lactobacillus | 59 | Healthy-Urine |
| Unknown (-299.077321242328) | Lactobacillus | 48 | Healthy-Urine |

Significant positive interactions were determined by pairwise Pearson correlations between OTUs and metabolites significantly enriched in the respective groups (i.e. Healthy-Urine). All FDR-corrected p-values are < 0.05, and r > 0.6. Only showing interactions with >10 No. of OTUs/metabolite. Metabolites are listed with their mass:charge (m/z) ratios. Negative values indicate metabolites identified in negative electron spray ionization mode, whereas positive values indicate metabolites identified in positive mode. Group indicates cohort (healthy or USD) and the sample type (urine or stool).

### Discussion

The objective of the current study was to take a multi-site, multi-omics approach to define dysbiosis in a representative population of patients with an active episode of USD, determine which site of microbial activity was most relevant for USD, and which microbe-metabolite interactions may be promoting or inhibiting stone growth.

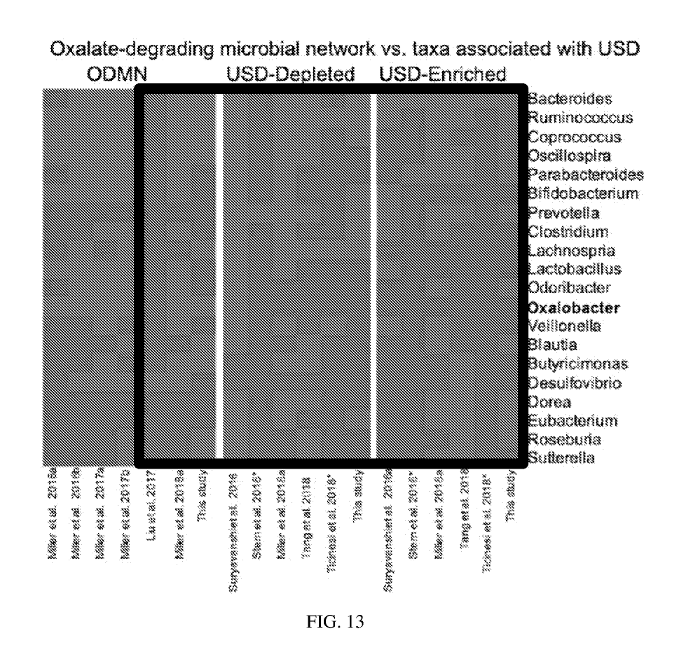

The results of the current study for the gut microbiota, are largely in congruence with past metagenomic studies (FIG. 13), indicative of a consistent form of dysbiosis. Specifically, meta-analysis of these studies in conjunction with those focused on the community of bacteria associated with oxalate metabolism finds that the taxa reduced in the gut microbiota of USD patients largely overlaps with the oxalate-degrading microbial network (FIG. 13, Table 13). Furthermore, in congruence with metagenomic studies but in contrast to many studies that determined O. formigenes colonization through culture-based or PCR-based methods, no difference in O. formigenes colonization between healthy and USD cohorts was found, nor was a negative correlation between O. formigenes colonization and antibiotic use found (FIG. 10). It is not currently known why there is this difference in metagenomic and culture- or PCR-based methods of detection.

Table 13

OTUs from the stool microbiota that exhibit a significant Pearson correlation with the relative abundance of O. formigenes

| Lowest Assigned Taxonomy | Taxonomic Level | No. of OTUs | r | FDR |
|---|---|---|---|---|
| | | | | <0.001- |
| Clostridiales | Order | 18 | 0.43-0.8 | 0.028 |
| Ruminococcaceae | Family | 14 | 0.42-0.7 | <0.001-0.03 <0.001- |
| Bacteroides | Genus | 11 | 0.42-0.59 | 0.039 |
| Faecalibacterium | Genus | 5 | 0.41-0.57 | <0.001-0.45 |
| Ruminococcus | Genus | 4 | 0.43-0.73 | <0.001-0.03 |
| Rikenellaceae | Family | 3 | 0.42 | 0.04 |
| Coprococcus | Genus | 3 | 0.47-0.52 | 0.001-0.008 |
| YS2 | Order | 2 | 0.58 | <0.001 |
| Clostridiaceae | Family | 2 | 0.45-0.48 | 0.005-0.017 |
| Lachnospiraceae | Family | 2 | 0.45-0.48 | 0.005-0.017 <0.001- |
| Roseburia | Genus | 2 | 0.49-0.53 | 0.004 |
| Oscillospira | Genus | 2 | 0.58-0.8 | <0.001 |
| RF39 | Order | 2 | 0.82 | <0.001 |
| Brevibacterium | Genus | 1 | 0.45 | <0.001 |
| Butyricimonas | Genus | 1 | 0.84 | <0.001 |
| Flavobacterium | Genus | 1 | 0.44 | 0.019 |
| Staphylococcus | Genus | 1 | 0.54 | <0.001 |
| Blautia | Genus | 1 | 0.45 | 0.015 |
| Lachnospira | Genus | 1 | 0.46 | 0.01 |
| Phascolarctobacterium | Genus | 1 | 0.61 | <0.001 |
| Sutterella | Genus | 1 | 0.45-0.48 | 0.014 |
| Oxalobacter | Genus | 1 | 1 | <0.001 |

The current study is the first metagenomics study to compare the urinary tract microbiome between USD and healthy populations. Several lines of evidence in this study point to the urinary tract microbiome as a greater contributor to the onset of USD than the gut microbiota. First, statistical analysis of the microbiota composition reveals that the urinary tract microbiota, but not the gut microbiota, was significantly different by USD-status (FIGS. 9a,b, Table 9). These results are corroborated by differential abundance analysis that showed a greater proportion of OTUs from the urinary tract were significantly different between the USD and healthy cohorts compared to the gut (FIGS. 9c,d, Table 10). Secondly, the urinary tract microbiota composition, but not the gut microbiota was also significantly different based on antibiotic use, family history of USD, and sex (Table 9). These factors have all been associated with USD in the past. Third, the overlap between the taxonomic profile of the kidney stone microbiota and that of the urine from USD patients was much greater than the taxonomic profile of the urine between the healthy and USD cohorts (FIG. 6). This result raises the possibility that bacteria in the urinary tract of people at risk for USD may play a direct role in stone formation. Finally, by integrating the microbiome data with the urinary metabolome data, it has been found that what differentiates the USD and healthy cohorts the most is the microbe-metabolite networks of the urinary tract microbiome and urinary metabolome. The metabolome is the end result of human and microbe metabolic processes. Furthermore, the urinary metabolome specifically is a known risk factor for USD that is often targeted in metabolic analyses. Thus, integration of microbiome and metabolome data allows for the honing in on the most important microbe-metabolite interactions for USD. Specifically here, the association between Lactobacillus bacteria and three currently unknown metabolites was the most differentiating factor from the integrated datasets (Tables 10 and 12). However, bacteria from the Enterobacteriaceae family had a strong association with the urinary tract of the USD cohort (Table 10). Interestingly, while many clinics often perform bacterial analysis on urine and kidney stones, the instant results show a strong bias of culture results compared to when DNA is extracted directly from stone or urine samples (FIGS. 6, 8, and 14). Specifically, there was an apparent culture bias against the Firmicutes, Actinobacteria, and rare phyla (FIG. 6). Importantly, the Lactobacillus, which were found to be greatly reduced in the USD population, is part of the Firmicutes phylum. Thus, culture-based approaches to microbial profiling in the urine and kidney stones may overlook important features of the urinary tract microbiome in association with USD, as has been noted previously.

Evidence is increasingly mounting as to the health-protective and disease promoting effects of urinary tract Lactobacillus and Enterobacteriaceae, respectively. While patients with struvite stones were excluded from the current study, a strong association with Enterobacteriaceae in the urine of the USD cohort was still found, indicating that bacteria from this family may generally promote stone growth in the urinary tract.

Urinary stone disease represents diverse pathologies, likely with equally diverse causal mechanisms that lead to stone formation. In the current study, patients were recruited with different stone types that included calcium oxalate, calcium phosphate, uric acid stones, and some composite stones, specifically to determine if there was an underlying association between dysbiosis with the microbiome and the onset of USD. While the results strongly suggest a common dysbiotic link between the microbiome and different pathologies of USD, it is likely that the specific groups of bacteria lost/gained in the gut or urinary tract contributes to the type of stone that manifests in the patient.

To conclude, the current study provides the most direct and proximate link between antibiotic use, the microbiome, and USD. Results of the study provide strong evidence for a combination of loss and gain of function dysbiosis centered on oxalate metabolism in the gut and Lactobacillus/Enterobacteriaceae in the urinary tract.

### Methods

### Recruitment of Participants

Patients who had an active episode of USD were given the option to participate in the current study. Control subjects without a history of USD were recruited by the clinical research unit (CRU) at Cleveland Clinic. All subjects were required to fill out a questionnaire detailing information associated with health, diet, and use of medications. Exclusion criteria included prior personal history of USD (healthy cohort only), chronic gastrointestinal issues, and age (<18 years old). Patients were not excluded on the basis of diet, age (>18 years old), or medications (antibiotics or otherwise) in order to test hypotheses associated with factors that impact the microbiome in ways that could facilitate the onset of USD. The prospective clinical cohort were representative of the typical USD population relative to stone composition, age, and presence of co-morbidities (Table 8).

### Sample Collection and Processing

Each subject was asked to provide a stool sample and a voided urine sample. Stool samples were self-collected by study subjects using a provided rectal swab containing modified Cary-Blair medium. Voided, clean-catch mid-stream urine was collected from all subjects, either in clinic or in the preoperative area prior to the stone procedure and pre- or perioperative antibiotics. From the urine sample in culture & sensitivity preservative (BD Scientific), 200 ul was used for cell culture and the remainder was used for DNA extraction. Urine, stool, and stone samples were stored in preservative at 4° C. prior to processing within 24 hours of collection.

Stone samples were collected after surgical procedure for removal (uteroscopy or percutaneuous nephrolithotomy), with a portion of the sample sent for clinical analysis of composition. Remaining stone samples were rinsed with sterile PBS to remove potential host bacteria contamination, flash frozen in liquid nitrogen and pulverized with a sterile mortar and pestle. Half of the pulverized stone was suspended in 15% glycerol and stored at -80° C. before culturing and the remainder of the pulverized stone was used for DNA extraction.

For cultures, 100 uL of urine or stone powder submerged in glycerol was inoculated to Columbia Blood Agar (Edge Biologicals, Memphis, TN) and MacConkey’s agar (Oxoid Agar Biological, ThermoFisher Scientific) plates. Bacteria were incubated aerobically at 37° C. for up to 5 days and were monitored daily for growth. Colonies were picked using a flame sterilized loop and suspended in 1 mL PBS. Culture conditions were designed to mimic culture conditions in typical clinical practices.

Urine, urine culture, and stone culture DNA was isolated using the Urine DNA Isolation Kit for Exfoliated Cells or Bacteria (Norgen, Thorold, ON, Canada). Prior to extraction, the urine sample was centrifuged 15,000 g for at least five minutes and the culture samples in PBS were centrifuged at 14000 g for three minutes. Pellets were re-suspended and mixed with 600 ul lysis buffer B, 12 ul lysozyme stock, 10 ul Proteinase K, and 20 ul mutanolysin. The mixture was incubated at 37° C. for 60 minutes, with vortexing every 15 minutes. The remainder of the protocol was followed according to the manufacturer’s instructions.

To ensure consistent extraction of DNA from kidney stones, a modified protocol from the Qiagen DNeasy Blood & Tissue Kit was developed (QIAGEN GmbH, Hilden, Germany). Specifically, buffer ATL was added to cover a pulverized stone sample and incubated with lysozyme, mutanolysin, and protease K at 37° C. for 1 hour, vortexing every 15 minutes. Subsequent processing was performed according to the manufacturer’s protocol.

Approximately 0.25-1 g of fecal samples were recovered from fecal swabs after centrifugation and collection of the pellet. QIAamp PowerFecal DNA Kit (QIAGEN GmbH, Hilden, Germany) was used for DNA extraction. For all DNA extractions, negative controls that included sterile water first placed into collection vessels and all extraction reagents were performed in conjunction with every round of extractions. Subsequently, all extractions were verified with gel electrophoresis and concentrations were quantified with a Nanodrop Spectrophotometer (Thermo Scientific). Only samples that exhibited the presence of a band on gel electrophoresis and had a DNA concentration >15 ng/ul were submitted for sequencing. No negative controls from any preparation had any quantifiable DNA. While all stool samples had quantifiable DNA, only 64 of the 67 urine samples and 10 of the 15 stone samples had detectable DNA that was used in downstream sequencing.

Urine samples were prepared for untargeted metabolomics by diluting each sample 1:4 in a 50% acetonitrile solution containing two internal standards, 30 uM 4-nitrobenzoic acid (Acros Organics, Fair Lawn, NJ, USA), and 2 uM debrisoquine (Santa Cruz Biotechnology, Dallas, TX, USA). Samples were centrifuged at 18,000 rcf for 5 minutes to precipitate proteins, and the supernatant was recovered and stored at -80° C. prior to analysis.

### DNA Sequencing and Analysis

Extracted DNA from feces, urine, and kidney stones was sent to Argonne National Laboratory (Chicago, IL) for sequencing of the V4 region of the 16 S rRNA gene on the Illumina MiSeq platform after amplification with the 515 F and 806 R primers. Barcodes with 12 base pairs were added to the amplified region and samples multiplexed on a single lane for 150 base pair, paired-end sequencing.

Raw sequencing data were demultiplexed and quality-controlled with default parameters in QIIME. Operational taxonomic units (OTUs) were assigned with open reference assignment, with 97% homology compared to a reference database composed of the Greengenes dataset and from previous datasets of de novo assigned OTUs, to permit direct comparison across studies. All OTUs that did not exhibit a match from the reference database were classified de novo. Sequences associated with chloroplasts, mitochondria, chimeras, or that had <10 representations across the dataset for each sample type were removed prior to downstream analyses, as previously described.

Data were normalized with a negative binomial Wald test through the DESeq2 algorithm prior to diversity analyses. For α-diversity, Margalef’s species richness, equitability, Shannon’s Index, and Phylogenetic diversity were quantified. Statistical analysis of α-diversity was calculated through paired t-tests in R statistical software89. For β-diversity, both weighted and unweighted UniFrac distances were calculated and statistical analysis was conducted through a Permanova analysis (Adonis), with 999 permutations. Differential abundance analysis was conducted through a Wald test, which determines signifiscance through the log2 fold change of normalized OTU abundance between groups, divided by standard error. The p-values were then adjusted to account for false discoveries (FDR). The network of bacteria that co-occur with Oxalobacter formigenes was performed as previously described. Briefly, the relative abundance of OTUs was correlated to the relative abundance of O. formigenes, using FDR-corrected Pearson correlations.

### Untargeted Metabolomics and Analysis

Urine from 18 USD subjects and 31 control subjects was available for metabolomic analysis. After the samples were prepared as above, they were submitted for processing via liquid chromatography/tandem mass spectroscopy (LC-MS-MS). External standards were added to the samples prior to injection onto the Vanquish UHPLC system coupled to a Q Exactive HF hybrid quadrupole-orbitrap mass spectrometer (Thermo Scientific, Waltham, MA). The mass spectrometer was operated in positive and negative electrospray ionization modes over a mass range of 50-750 Da. The XCMS software package was used to de-convolute the raw data. The detected ions were normalized to total creatinine and further analyzed using Metabolyzer software. Concentrations were quantified with comparison to the two internal standards added at a known concentration. All metabolites were defined by ionization, molecular mass, and retention time (m/z) in the UHPLC-MS-MS system. Metabolites were given putative identification by comparison with metabolites in the KEGG, HMDB, LIPIDMAPS, and BioCyc databases.

### Integration of 16S rRNA and Metabolomic Data

To integrate the 16 S rRNA and metabolomic data, OTUs significantly enriched in the 1) urine of the healthy group; 2) urine of USD group; 3) stool of the healthy group; or 4) stool of the USD group were used. Normalized counts of these OTUs were integrated with the significantly different metabolites from the 1) urine of the healthy group; or 2) urine of the USD group. Correlation networks were calculated by conducting all pairwise microbe-metabolite Pearson correlations. Only correlations >0.6 and with an FDR-corrected p-value <0.05 were used in downstream analyses. Correlation networks were generated from: 1) The urine microbiome and urine metabolome of the healthy group; 2) The urine microbiome and urine metabolome of the USD group; 3) The fecal microbiome and urine metabolome of the healthy group; and 4) The fecal microbiome and urine metabolome of the USD group. Resulting microbe-metabolite networks were visualized in Cytoscape.

### Example 4:Antibiotic Use and the Gut Microbiota

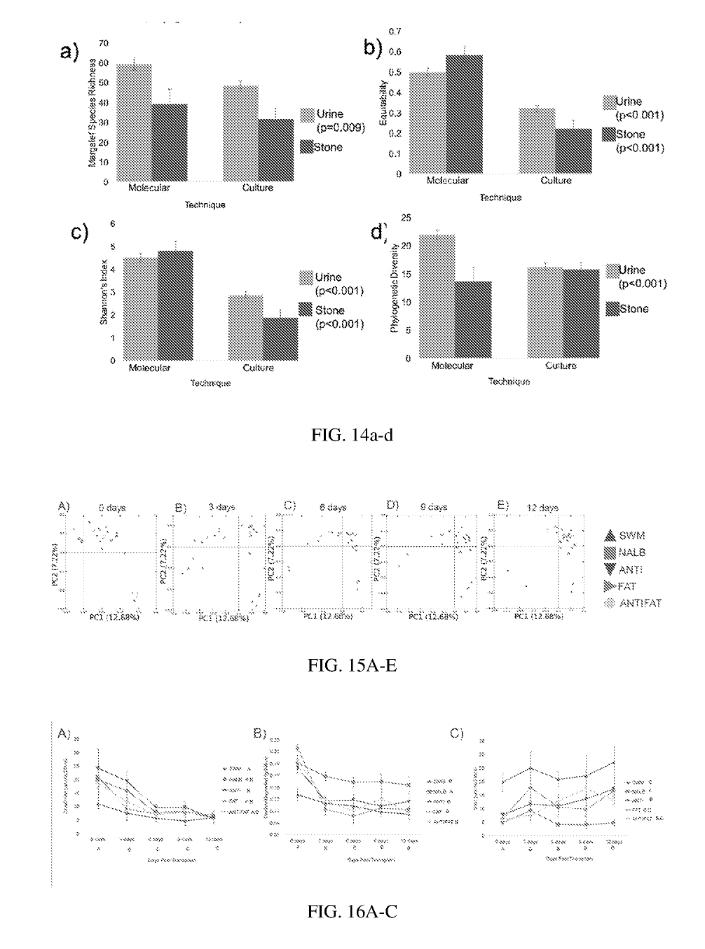

Microbial transplants were given from Neotoma albigula to Swiss-Webster mice. Following the transplants mice were given either nothing (NALB), antibiotics (ANTI), a high fat, high sugar diet (FAAT), or both (ANTIFAT). Swiss-Webster mice given their own feces were also used (SWM) as a negative control. Beta-diversity by group-time. FIG. 15 shows PCoA plots that are based upon the unweighted UniFrac distance matrices for all groups and timepoints. Points are only shown for each individual time-point in order to show how each group changes over time. The plots are labeled with the number of days post-transplant: 15A) 0 days; 15B) 3 days; 15C) 6 days; 15D) 9 days; and 15E) 12 days. The data shows that antibiotic use causes a transient shift in the gut microbiota, which returns back to the post-transplant baseline, rather than the baseline before the transplant is carried out.

FIG. 16 shows urinary/oxalate metrics after antibiotic and/or diet treatment as indicated in FIG. 15. Each time point represents the average daily value for the 3-day interval. FIG. 16A) shows urinary creatinine excretion; FIG. 16B) shows total microbial oxalate metabolism (oxalate consumed minus oxalate excreted); and FIG. 16C) shows urinary oxalate excretion (Urox). The letters indicate statistically significant differences either by Treatment group (in legend) or by time point (on x-axis) as determined by a repeated measures ANOVA and post-hoc Tukey’s honestly significant difference analysis.

### Example 5: Effect of Formate-Degrading Bacteria on Oxalate Metabolism

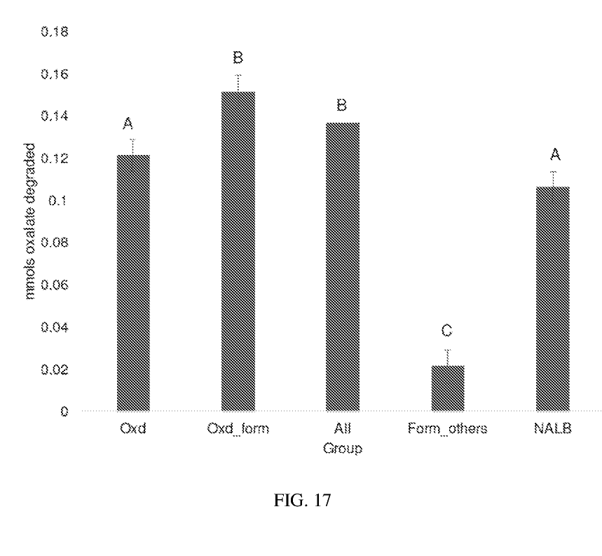

As seen in FIG. 17, different combinations of bacteria were grown in the presence of 50mM oxalate to quantify differences in oxalate metabolism between the groups. The groups were as follows (from left to right): 1) oxalate-degrading bacteria alone (Bacteroides vulgatus); 2) oxalate- and formate-degrading bacteria (Bacteroides vulgatus & Enterobacter sp.); 3) all functional groups listed in Table 2) all functional groups listed in Table 2 minus the oxalate-degrading bacteria; 5) the whole N. albigula community. The letters A, B, and C indicate statistical groups.

The results show that adding formate-degrading bacteria to oxalate-degrading bacteria does by itself significantly increase oxalate metabolism. Furthermore, the levels of oxalate degradation in the combination groups were significantly higher than for the N. albigula whole microbiota.

The complete disclosure of all patents, patent applications, and publications, and electronically available material cited herein are incorporated by reference. The foregoing detailed description and examples have been given for clarity of understanding only. No unnecessary limitations are to be understood therefrom. Although the invention has been described with reference to several specific aspects, the invention is not limited to the exact details shown and described, for variations obvious to one skilled in the art will be included. The description is not meant to be construed in a limited sense. Various modifications of the disclosed aspects, as well as alternative aspects of the inventions will become apparent to persons skilled in the art upon the reference to the description of the invention. It is, therefore, contemplated that the appended claims will cover such modifications that fall within the scope of the invention.

What is claimed is:

1. A method of determining the risk that a subject will develop urinary stone disease (USD) or hyperoxaluria, comprising
conducting a differential abundance analysis of the bacteria present in a stool and/or urine sample obtained from the subject;
determining a ratio of bacteria associated with health to bacteria associated with USD or hyperoxaluria present in the subject’s stool and/or urine sample;
and assigning a level of risk for developing USD or hyperoxaluria based on the ratio.

2. The method of claim 1, wherein the method further comprises determining the type of bacteria associated with heath that are either missing or diminished in the subject’s stool and/or urine sample and administering a composition comprising one or more of the missing or diminished types of bacteria to the subject.

3. A method of decreasing the risk that a subject will develop urinary stone disease (USD) or hyperoxaluria, comprising
conducting a differential abundance analysis of the bacteria present in a stool and/or urine sample obtained from the subject;
determining the bacteria associated with heath that are either missing or diminished in the subject’s stool and/or urine sample; and
administering to the subject a composition comprising one or more of the missing or diminished bacteria.

4. The method of claim 3, wherein the one or more missing or diminished bacteria are selected from the following: (i) one or more species of bacteria falling under the genus Ruminococcus; (ii) one or more species of bacteria falling under the genus Enterobacter; (iii) Bacteroides acidofaciens; (iv) Bacteroides vulgatus; (v) Bacteroides dorei; (vi) Oxalobacter formigenes; (vii) Methanobrevibacter smithii; (viii) one or more species of bacteria falling under the genus Desulfovibrio; (ix) Bacteroides betaiotamicron; (x) Coprococcus comes; (xii) Lactobacillus helveticus; and (xiii) Lactobacillus plantarum.

5. The method of claim 4, wherein the one or more missing or diminished bacteria are selected from the following: (i) one or more species of bacteria falling under the genus Ruminococcus; (ii) one or more species of bacteria falling under the genus Enterobacter; (iii) Bacteroides acidofaciens; (iv) Bacteroides vulgatus; and (v) Bacteroides dorei.

6. The method of claim 4, wherein the one or more missing or diminished bacteria are selected from the following: Oxalobacter formigenes; Methanobrevibacter smithii; one or more species of bacteria falling under the genus Desulfovibrio; Bacteroides betaiotamicron; Coprococcus comes; Lactobacillus helveticus; and Lactobacillus plantarum.

7. The method of claim 3, wherein the composition further comprises a prebiotic.

8. The method of claim 7, wherein the prebiotic comprises oxalate.

9. The method of claim 3, wherein one or more of the missing or diminished bacteria are obtained from Neotoma albigula.

10. A method of guiding the treatment of urinary stone disease (USD) or hyperoxaluria in a subject, comprising:
conducting a differential abundance analysis of the bacteria present in a stool and/or urine sample obtained from the subject;
determining a ratio of bacteria associated with health to bacteria associated with USD or hyperoxaluria present in the subject’s stool and/or urine sample;
assigning a level of severity of USD or hyperoxaluria based on the ratio;
and providing treatment appropriate for the level of severity.

11. The method of claim 10, wherein the treatment comprises administering one or more types of bacteria associated with health to the subject.

12. The method of claim 11, wherein the one or more bacteria associated with health are selected from the following: (i) one or more species of bacteria falling under the genus Ruminococcus; (ii) one or more species of bacteria falling under the genus Enterobacter; (iii) Bacteroides acidofaciens; (iv) Bacteroides vulgatus; (v) Bacteroides dorei; (vi) Oxalobacter formigenes; (vii) Methanobrevibacter smithii; (viii) one or more species of bacteria falling under the genus Desulfovibrio; (ix) Bacteroides betaiotamicron; (x) Coprococcus comes; (xii) Lactobacillus helveticus; and (xiii) Lactobacillus plantarum.

13. The method of claim 12, wherein the one or more bacteria associated with health are selected from the following: (i) one or more species of bacteria falling under the genus Ruminococcus; (ii) one or more species of bacteria falling under the genus Enterobacter; (iii) Bacteroides acidofaciens; (iv) Bacteroides vulgatus; and (v) Bacteroides dorei.

14. The method of claim 12, wherein the one or more bacteria associated with health are selected from the following: Oxalobacter formigenes; Methanobrevibacter smithii; one or more species of bacteria falling under the genus Desulfovibrio; Bacteroides betaiotamicron; Coprococcus comes; Lactobacillus helveticus; and Lactobacillus plantarum.
